(19) United States
(12) Patent Application Publication
(10) Pub. No.: US 2003/0211076 A1
(43) Pub. Date: Nov. 13, 2003

(54) Compositions and methods for treatment of proliferative disorders

(76) Inventors: Wande Li, Malden, MA (US); Herbert M. Kagan, Arlington, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/275,643

(22) PCT Filed: May 10, 2001
(86) PCT No.: PCT/US01/15191

(51) Int. Cl. A61K 38/44; A61K 38/19; A61K 33/34

(52) U.S. Cl. 424/85.1; 424/94.4; 424/638

(57) ABSTRACT

The present invention relates to compositions and methods for preventing and treating AIDS, AIDS-malignancy, and other tumors. In particular, this invention comprises modulation of mitogenic and angiogenic growth factors with lysyl oxidase and its homologues. In other aspects of this invention, lysyl oxidase and its homologues provide angiogenic inhibition to the transactivating protein Tat of HIV-1.

### CROSS REFERENCE TO RELATED APPLICATIONS

This application claims the priority benefit of provisional U.S. Patent Application No. 60/202,568, filed May 10, 2000 entitled, LYSYL OXIDASE AS A BIOLOGICAL SUPPRESSOR OF THE MITOGENIC AND TUMORIGENIC ACTIVITIES OF CELL MITOGENS, the whole of which is hereby incorporated herein by reference.

### STATEMENT REGARDING FEDERALLY SPONSORED RESEARCH OR DEVELOPMENT

Part of the work leading to this invention was carried out with United States Government support provided under a grant from the National Institutes of Health, Contract Number HL 13262. Therefore, the United States Government has certain rights in this invention.

### BACKGROUND OF THE INVENTION

Growth factors and angiogenic factors regulate the proliferation, migration and differentiation of mammalian cells, playing important roles in embryogenesis, angiogenesis and wound healing. If the mitogenic and angiogenic potential of cells is not properly regulated, then the abnormal proliferation of cells can cause the development of conditions such as tumors and cancers.

Various types of cancers and tumors developed in humans and in patients with acquired immunodeficiency syndrome (AIDS) are leading causes of human disability and death in the United States. Current treatment of cancerous tumors includes surgical removal, which is usually accompanied by chemotherapy and/or radiation. Such treatment regimen can be debilitating and often requires extensive post surgical care. Although these traditional approaches for cancer treatment can be successful in the management of selected groups of solid tumors and cancers, many malignant tumors remain resistant to traditional approaches, and the prognosis in such cases is correspondingly grave.

Simultaneously, AIDS and AIDS-associated malignancies are among the gravest current threats to public health. In particular, Kaposi's sarcoma (KS), an angioproliferative condition, is frequently associated with human immunodeficiency virus type 1 (HIV-1)-infected patients suffering from AIDS. The flat, plaque or nodular lesions of KS present mainly on the skin are predominantly characterized by the appearance of spindle-shaped cells with markers of endothelial origin associated with infiltration of inflammatory cells and with angiogenesis. AIDS-associated KS (AIDS-KS) is the most aggressive form, which not only involves the skin and mucosa but also the viscera. Although highly active anti-retroviral therapy (HAART) has prolonged the survival time in some AIDS patients, almost all patients with AIDS-KS eventually develop widely disseminated disease, which is resistant to conventional therapies leading to increased morbidity and mortality. AIDS patients with pulmonary KS have the poorest prognosis, with median survival times of less than six months.

Under physiological conditions, the balance between growth factors and growth inhibitors is critical to the maintenance of homeostasis of tissues and organs. The growth pattern of tumors and cancer cells is characterized by an uncontrollable multiplication. This abnormal cellular proliferation may result from imbalances between growth factors and biological modes of control of their productions and/or their functions. The activation by growth factors of para- and/or autocrine mechanisms and the activation by angiogenic molecules of angiogenesis confer a growth advantage and thus contribute to the malignant potential of tumor cells as exemplified by those with AIDS-associated malignancy. It is widely accepted that disruption of para- and/or autocrine loops of growth factors and inhibition of angiogenesis are two major strategies of anti-tumorigenesis. Thus, the discovery of potent inhibitors that selectively inactivate key growth factors and angiogenic molecules is an attractive approach for the development of tumor and AIDS therapy.

### BRIEF SUMMARY OF THE INVENTION

The present invention is directed to the use of an enzyme that has been discovered to have the ability to modulate biological agents that regulate a variety of cellular processes, e.g., proliferation, migration, and differentiation in both normal and abnormal cells. Accordingly, in one aspect, this invention provides for compositions and methods for prophylaxis and treatment of a condition associated with abnormal cellular proliferation by targeting mitogenic and angiogenic factors, including a transactivator for the replication of HIV-1. The enzyme of the present invention is lysyl oxidase (LO), including homologues of LO.

In one embodiment, the invention includes a therapeutic composition for prophylaxis and treatment of a condition associated with an abnormal cellular proliferation comprising an effective amount of a therapeutically active portion of an inhibitor of cell growth. Administered in a pharmaceutically acceptable inert carrier substance, the inhibitor oxidizes cell growth factors at lysine residues. The amount of inhibitor provided is effective in preventing and treating a condition associated with such abnormal cellular proliferation. In one aspect, the abnormal cellular proliferation includes, inter alia, tumors, lesions and wounds.

A further embodiment of the present invention includes the use of LO as a therapeutic composition for prophylaxis and inhibition of angiogenesis. Administered in a pharmaceutically acceptable inert carrier substance, the active portion of LO and/or its homologue is an angiogenic inhibitor of both normal and tumor cells. In one aspect, the angiogenic inhibitor oxidizes and inactivates the angiogenic factors at lysine residues.

In a further embodiment of the present invention, the active portion of LO and/or its homologues is used in a therapeutic composition for prophylaxis and inhibition of a condition associated with a microorganism infection. In one aspect, the inhibitor is administered in a pharmaceutically acceptable inert carrier substance in an effective amount. In another aspect, the inhibitor inactivates a transactivator for replication of this microorganism by oxidizing the transactivator at lysine residues. This allows for an effective inhibition of microorganism replication.

In another aspect, the condition associated with a microorganism is AIDS and the microorganism is the HIV-1 virus. In a particularly preferred embodiment, the transactivator for the replication of said microorganism is HIV-1 Tat.

In a further embodiment, for the compositions of the invention, the inhibitor is LO or a homologue of LO. LO may be purified from bovine connective tissue or human tissue. In another embodiment, the therapeutic composition of the present invention can also be co-administered with compounds such as antineoplastic agents, cytokines, hormones, and/or copper ions (Cu²⁺) for the modulation of endogenous and exogenous LO.

As another embodiment of the present invention, the conditions associated with such therapeutic treatments include, inter alia, breast cancer, colon cancer, renal cancer, prostate cancer, ovarian cancer, lung cancer, brain cancer, skin cancer, embryo carcinoma, teratocarcinoma, germ cell tumor, uterine cancer, osteocarcoma, fibrosacoma, melanoma, and AIDS-associated malignancies, including Kaposi's sarcoma, angiogenic diseases and tumors such as neovascular glaucoma, angiomatosis, hemangioma, angiofibroma, angioendothelioma and hyperplastic diseases with or without inflammations such as thyroid hyperplasia and giant cell arthritis. In one aspect, an aberrant cell growth can be derived from a mammal, preferably the mammal is human, and can consist of a tumor, lesion, or wound.

In a further embodiment, the present invention includes a kit comprising the inhibitors described above and instructions for use thereof.

In a further embodiment, the present invention includes a method of treating a patient believed to be at risk of suffering from a disease associated with abnormal cellular proliferation. The method comprises providing such patient with an administration of an effective amount of the therapeutic composition of LO and/or its homologues.

In another embodiment, the present invention also includes a method of modulating cell proliferation and angiogenesis. In one aspect, such modulation can be performed by contacting and inactivating mitogenic and angiogenic factor with an inhibitor in an effective amount to modulate cell proliferation and determining whether or not cell proliferation is regulated.

In another embodiment, the present invention also includes a method of treating a patient believed to be at risk of suffering from a condition associated with a microorganism infection. In one aspect, the microorganism is HIV-1. In another aspect, such method comprises inhibiting the activity of a transactivator involved in the replication of such microorganism. In a further aspect, the transactivator is inhibited by oxidation of lysine residues. In an another aspect, the transactivator for the replication of said microorganism is HIV-1 Tat protein.

In another embodiment, the methods described above are related to the following disease conditions: breast cancer, colon cancer, renal cancer, prostate cancer, ovarian cancer, lung cancer, brain cancer, skin cancer, embryo carcinoma, teratocarcinoma, germ cell tumor, uterine cancer, osteocarcoma, fibrosacoma, malanoma, and AIDS-associated malignancies including Kaposi's sarcoma, angiogenic diseases and tumors such as neovascular glaucoma, angiomatosis, hemangioma, angiofibroma, angioendothelioma and hyperplastic diseases with or without inflammations such as thyroid hyperplasia and giant cell arthritis.

In a further embodiment, in the methods described above, the cell growth inhibitor is administered by a physiologically compatible vehicle. In another embodiment, the methods comprise of the additional step of determining treatment progress by, for example, tissue biopsy.

### BRIEF DESCRIPTION OF THE FIGURES

Other features and advantages of the invention will be apparent from the following description of the preferred embodiments thereof and from the claims, taken in conjunction with the accompanying drawings, in which:

FIG. 1 shows a comparison of the amino acid sequence at the C-terminal regions of the various isoforms of VEGF A, specifically, VEGF121, VEGF145, VEGF165, VEGF189, and VEGF206, highlighting the lysine (K) residues;

FIG. 2 shows an amino acid sequence of the Tat protein;

FIG. 3 shows a comparison of the amino acid sequence highlighting the lysine and arginine-rich basic domain in the C-terminal region of Tat, FGF2, VEGF165, and VEGF189;

FIG. 4 depicts hydrogen peroxide (H₂O₂) release in the reaction of varying amounts of lysyl oxidase (LO) with FGF2 in vitro, using a HRP-coupled fluorescence assay. H₂O₂ release increases with increasing concentration of FGF2 incubated at 55° C., as shown in traces (a), (b), and (c), and incubated at 37° C. as shown in trace (d);

FIG. 5 is a photomicrograph of a gel showing LO-catalyzed crosslinking of ¹²⁵1-labeled FGF2 revealed by SDS-PAGE (sodium dodecyl sulfate polyacrylamide gel electrophoresis) and autoradiography. Lane 1 depicts ¹²⁵I-labelled FGF2 alone and lane 2 depicts ¹²⁵1-labelled FGF2 incubated with LO;

FIGS. 6A-6C depict a bar graph of LO modulation of FGF2-stimulated cell cycle progression revealed by FACS analysis. FIG. 6A shows dose response to FGF2 of cell cycle progression. FIG. 6B shows LO inhibition of FGF2-stimulated cell cycle progression. FIG. 6C shows the effects of inactivation of LO by BAPN on FGF2-stimulated cell cycle progression;

FIG. 7 depicts an immunoblot of LO inhibition of FGF2-stimulated MAP kinase phosphorylation in growth-arrested Swiss 3T3 cells treated with various combinations of FGF2, LO, BAPN and genistein;

FIGS. 8A-8F show photomicrographs of nuclear localization of FGF2 in growth-arrested Swiss 3T3 cells treated with FGF2, LO, BAPN and their combinations;

FIGS. 9A-9B is a bar graph of LO inhibition of tumor cell (NIH 3T3 6-1) growth. FIG. 9A shows inhibition of cell growth with increasing doses of LO. FIG. 9B shows the effect of inactivation of LO by BAPN;

FIGS. 10A-10F show numerical and morphological alterations in tumor cells treated with LO and/or BAPN revealed by phase contrast microscopy (25×);

FIGS. 11A-11C show Western blotting of SDS-PAGE electrophoretograms of intracellular FGF2 crosslinking by LO in NIH 3T3 and NIH 3T3 6-1 cells treated without (FIG. 11A) or with various combination of LO and/or BAPN (FIG. 11B). FIG. 11A shows FGF2 expression in NIH 3T3 cells (lane 1) and in NIH 3T3 6-1 cells (lane 2) in the absence of additives. FIG. 11B shows forms of FGF2 in NIH 3T3 6-1 cells treated without (lane 1) or with LO (lane 2) or LO inactivated by BAPN (lane 3). FIG. 11C shows total protein profiles in the control and treated cells in each lane corresponding to those in FIG. 11B revealed by Coomassie blue staining of SDS-PAGE electrophoretograms;

FIGS. 12A-12B are graphs of H₂O₂ release in the reaction of Tat with LO in vitro. FIG. 12A shows a dose and temperature-dependent response where the solid curves represent H₂O₂ release at 55° C. and where the dotted curves represent H₂O₂ release at 37° C. FIG. 12B represents the effects of BAPN on the reaction of Tat with LO;

FIGS. 13A-13C depict LO inhibition of the growth of Tat-stimulated porcine vascular endothelial cells (VEC). FIG. 13A shows a dose response of Tat-stimulated cell growth. FIG. 13B shows LO inhibition of Tat-stimulated cell growth. FIG. 13C shows the effects of inactivation of LO by BAPN on Tat-stimulated cell growth;

FIGS. 14A-14F show numerical and morphological alterations in Tat-stimulated VEC cells treated with LO revealed by phase-contrast microscopy (25×). The VEC cells are treated with combinations of Tat, LO and BAPN;

FIGS. 15A-15F show inhibition by LO of angiogenic response of Tat-stimulated VEC on Matrigel revealed by phase-contrast microscopy (25×). The VEC cells are treated with combinations of Tat, LO and BAPN;

FIGS. 16A(A-D) show a six-hour incubation of VEC on Matrigel showing angiogenic inhibition by LO as photographed by phase-contrast microscopy (25×). The cells are incubated with combinations of LO and BAPN;

FIGS. 16B(A-D) show a twenty-four-hour incubation of VEC on Matrigel showing angiogenic inhibition by LO as photographed by phase-contrast microscopy (25×). The cells are incubated with combinations of LO and BAPN;

FIGS. 16C(A-D) show a forty-eight-hour incubation of VEC on Matrigel showing angiogenic inhibition by LO as photographed by phase-contrast microscopy (25×). The cells are incubated with combinations of LO and BAPN;

FIG. 17 shows a graph of H₂O₂ release in the reaction of LO with VEGF165 in vitro;

FIGS. 18BA-18C show inhibition by LO of the growth of VEGF165-stimulated VEC. FIG. 18A shows a bar graph of dose response of VEGF165-stimulated cell growth. FIG. 18B shows a bar graph of LO inhibition of VEGF165-stimulated cell growth. FIG. 18C shows a bar graph of the effects of inactivation of LO by BAPN on VEGF165-stimulated cell growth;

FIG. 19 shows a graph of H₂O₂ release in the reaction of LO with PDGF-AB in vitro; and

FIGS. 20A-20C show inhibition by LO of PDGF-AB-stimulated cell cycle progression revealed by FACS analysis. FIG. 20A shows a bar graph of dose response of PDGF-AB in cell cycle progression. FIG. 20B shows a bar graph of LO inhibition of PDGF-AB stimulated cell cycle progression. FIG. 20C shows a bar graph of the effect of inactivation of LO by BAPN on PDGF-AB stimulated cell cycle progression.

### DETAILED DESCRIPTION OF THE INVENTION

According to the invention, lysyl oxidase (LO) and related molecules with or without catalytic activity may be used as therapeutic agents for the prevention and treatment of cancers/tumors, AIDS, AIDS-associated malignancy and other angiogenic and hyperplastic diseases by virtue of the ability of these molecules to catalytically inactivate or otherwise compete for the mitogenic, angiogenic and tumorigenic activities of cell mitogens and to inactivate HIV-Tat, a transactivator for the replication of HIV-1 and a pathological agent for the onset of AIDS-KS.

The recognized activity of LO according to the prior art is to oxidize specific lysine residues in collagen or elastin, thus inducing the crosslinking of these protein molecules to insolubilize and stabilize polymeric forms of these proteins in the extracellular matrix (ECM). LO, which plays a central role in the morphogenesis and repair of connective tissues of the cardiovascular, respiratory, skeletal and other systems of the body, is a copper-dependent enzyme expressed and secreted by fibrogenic cells, e.g., vascular smooth muscle cells (VSMC) and fibroblasts. In addition to its use of collagen and elastin as its major substrates, LO can also oxidize lysine residues in various globular proteins which have basic isoelectric points, such as histone H1 (Kagan et al., 1983). This enzyme has been identified in the tissues of numerous mammals, including human, bovine, rat and mouse. Abnormal LO activity in tissues and organs is implicated in various pathological states. LO deficiency is an important characteristic of several genetic disorders, e.g., Menkes syndrome in which the lack of the P-type ATPase in the membrane of secretory vesicles induces a defect in Cu efflux leading to abnormal accumulation of Cu in Menkes cells (Harris). In contrast, high levels of LO are associated with fibrotic diseases, e.g., atherosclerosis and lung and liver fibrosis (Kagan, 1986; Kagan et al., 1991). Thus, LO is an important enzyme for development and repair of the ECM and is involved in many human diseases.

LO initiates the covalent crosslinking of collagen or elastin in the ECM by oxidizing specific lysine residues to form peptidyl α-aminoadipic-δ-semialdehyde in these proteins. The resulting peptidyl aldehyde residues spontaneously condense with other peptidyl aldehydes or unreacted lysines to generate inter- and intra-molecular covalent adducts stabilizing the polymeric collagen or elastin fibers. In the reaction of LO with its substrates, hydrogen peroxide (H₂O₂) and ammonium are released in quantities stoichiometric with the peptidyl aldehyde product (Kagan, 1986; Kagan et al., 1991).

Primary mitogens and angiogens occurring in the extracellular space in a variety of tissues include fibroblast growth factors 2 (FGF2), 4 (FGF4), 5 (FGF5) and 6 (FGF6), vascular endothelial growth factor 165 (VEGF165) and platelet-derived growth factor-AB (PDGF-AB). The oxidation by LO of lysine residues in FGF2, PDGF-AB and VEGF165, for example, dramatically reduced their mitogenic potential thus inhibiting normal and tumor cell growth. Additionally, the Tat protein, encoded by human immunodeficiency virus type 1 (HIV-1), is a transactivator which possesses mitogenic/angiogenic properties. Nanomolar concentrations of LO markedly inhibited Tat-dependent proliferation and stimulation of angiogenesis of vascular endothelial cells. Thus, LO is a biological growth suppressor effectively modulating cell proliferation and angiogenesis and an inhibitor of Tat-dependent transactivation in HIV-1 infection through its interaction with specific mitogenic and angiogenic factors and HIV-1 Tat.

LO is synthesized by fibrogenic cells as a 46 kDa kDa proenzyme. Following signal peptide cleavage and N-glycosylation the resulting 50 kDa kDa proenzyme is secreted and then proteolytically cleaved to the 31±1 kD functional species in the extracellular space (Trackman et al., 1992; Li et al., 1995). The cDNA sequences of rat, mouse, chick and human LO reveal that the sequence coding for the propeptide is moderately (60-70%) conserved, whereas the sequence coding for the C-terminal 30 kDa kDa region of the proenzyme in which the active site is located is highly conserved (ca. 95%) (Kagan et al., 1995). It is well established that LO is a metalloenzyme requiring Cu²⁺ at its active site (Kagan, 1986; Kagan et al., 1991). The bovine aortic enzyme contains 1 mole of Cu²⁺ (II) per mature LO molecule (32 kD). In addition to Cu²⁺, LO also contains a covalently bound carbonyl cofactor recently identified as lysine tyrosylquinone (LTQ) deriving from crosslinking of Lys 314 to an oxidized derivative of Tyr 349 (Li et al., 2000). This peptidyl orthoquinone functions as a transient electron sink during amine oxidation (Kagan, 1986; Kagan et al., 1991).

The newly discovered activity of LO is that this enzyme can also function as a cell growth inhibitor. Thus, the cell growth inhibitor in accordance with the present invention comprises a biological growth suppressor that modulates cell proliferation and angiogenesis and also inhibits the transactivation of HIV-1. The cell growth inhibitor includes LO and LO homologues, preferably mammalian homologues. The inhibitor of the invention includes, inter alia, fragments and/or derivatives of LO and/or its homologues, with or without catalytic activity, recombinant LO, lysyl oxidase-like protein 1, and lysyl oxidase-like protein 2.

LO, purified from the connective tissue of bovine and other mammals, or its homologues or therapeutically effective portion thereof may be used as therapeutic agents in a method of treating any abnormal cellular proliferative or angiogenic state and HIV-1 infection. The therapeutically effective portion refers to a compound or composition effective to depress, suppress or inhibit mitogenesis, angiogenesis, or the transactivation effects of Tat. Such therapeutic agents include purified naturally occurring LO, human recombinant LO and catalytically active fragments (peptides) of LO. Without being bound by any theory, it is believed that the therapeutic agents would also include the binding domains for cytokines, growth hormones and cell surface adhesion molecules with the consensus sequence C-X₉-C-X-W-X₂₅₋₃₀-C-X₁₀₋₁₅ (C=cysteine, W=tryptophan, X=any amino acid) at the C-terminus of LO (e.g., the sequence in the human LO: CYDTYGADIDCQWIDITDVKPGNYILKVSVNPSYLVPESDYTNNVVRCDIRYTGHHA YASGC). This conserved sequence in LO is presumed to contribute to the molecular basis for the multiple-functions of this catalyst, such as regulation of cell proliferation and differentiation in addition to its central role in stabilization of the ECM. Thus, in achieving its therapeutic effect, LO may also directly bind to the cytokines, growth hormones, or adhesion molecules on the cell surface and interfere with the normal interaction between active ligands and their cognate receptors on the cell membrane by its competitive binding, as a result, altering cell behavior. Exemplary conditions with abnormal cellular proliferation may include, inter alia, breast cancer, colon cancer, renal cancer, prostate cancer, ovarian cancer, lung cancer, brain cancer, skin cancer, embryo carcinoma, teratocarcinoma, germ cell tumor, uterine cancer, osteocarcoma, fibrosacoma, melanoma. Exemplary conditions associated with varying angiogenic states include, inter alia, neovascular glaucoma, angiomatosis, hemangioma, angiofibroma, angioendothelioma and hyperplastic diseases with or without inflaminations such as thyroid hyperplasia and giant cell arthritis. Exemplary microorganism infection includes AIDS-associated malignancies such as, inter alia, Kaposi's sarcoma.

Substrates of LO

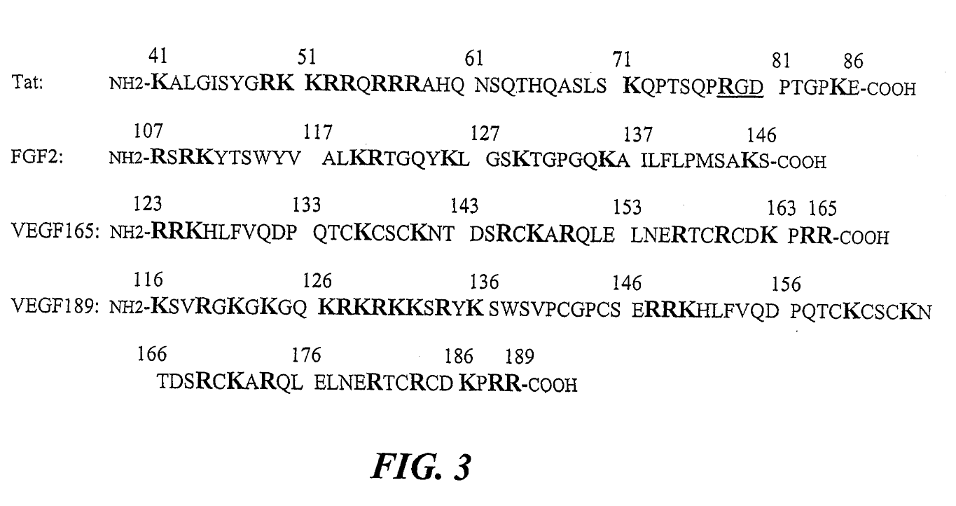

The newly discovered substrates of LO, including several key mitogenic/angiogenic factors and HIV-1 Tat as reported, see infra, share common chemical features and biological functions. These substrates contain peptidyl lysine residues, and are positively charged (basic) proteins with basic isoelectric points greater than 8.0. These factors appear in the extracellular space and thus are readily accessible to LO. These factors all target various types of cells including vascular endothelial cells (VEC), and stimulate their proliferation or angiogenesis. The factors compete and/or synergize with each other to exert their biological function. They all have a lysine-rich basic domain in their C-terminal regions (FIG. 3) and exhibit a high affinity for heparin and heparan sulfate proteoglycan (HSPG). They can be internalized by the cell and can be localized in the cell nucleus.

Fibroblast Growth Factors

Fibroblast growth factors (FGF) play a major role in proliferation, migration, and differentiation of cells occurring in all organs and solid tissues (Mason; Rifkin et al.; Nishimura et al.). This polypeptide is also implicated in a number of pathological states including wound healing, inflammation, angiogenesis and tumor growth (Rifkin et al.).

Human FGF2 is a basic protein (pI>9.0) containing for example in the 18 kDa species, 14 lysine residues in a total of 155 amino acids (Rifkin et al.; Burgess et al.). Certain of these lysines are accessible at the protein surface, among which K26, K125 and K135 (K=lysine) act as HSPG binding sites while K110 plays a critical role at the FGF2 receptor binding domain (residues 106-115) (Eriksson et al.; Faham et al.).

Human FGF2 is produced as four isoforms, i.e., 18, 22, 22.5 and 24 kDa molecules, that are translated from various initiation sites within a single mRNA species (Florkiewicz et al.; Prats et al.). These isoforms differ in their amino terminal extremities, which confer different intracellular localizations and functions (Florkiewicz et al.; Prats et al.; Stachowiak et al., 1996). The 18 kDa form is distributed in the cytoplasm and functions in cell growth and migration while the 22, 22.5 and 24 kDa forms are localized in the nucleus and appear to modulate processes in cell division (Stachowiak et al., 1996; Arese et al.; Bikfalvi et al.). Although FGF2 lacks a signal sequence as present in nascent proteins destined for secretion, evidence suggests that the 18 kDa isoform can be secreted by a pathway independent of the endoplasmic reticulum (Mignatti et al.).

FGF2 has been visualized in the ECM of different tissues (Rifkin et al.; Nugent et al.). Extracellular FGF2 forms a complex with heparan sulfate proteoglycan (HSPG), a major component of the ECM, thus stabilizing the growth factor (Schlessinger et al.). Moreover, membrane bound HSPG sites act as low-affinity receptors to provide a mechanism for ligand concentration and presentation of dimerized FGF2 to high affinity, tyrosine kinase receptors (FGFR 1-4) initiating several intracellular signaling cascades (Burgess et al.; Eriksson et al.). As a general mechanism, binding of FGF2 to cognate receptors induces receptor dimerization and autophosphorylation, leading to MAPK activation for cell division (Besser et al.). Recent evidence shows that internalized FGF2 coupled with its receptors in the cytoplasm or within the nucleus continues to provide growth signals to the cell (Sperinde et al.; Stachowiak et al., 1997). Peroxide appears to act as second messenger for FGF2 stimulation as evidenced by the observation that FGF2 elevated cellular H₂O₂ levels inducing c-fos expression (Lo et al.). H₂O₂ increases the affinity of FGF2 for its receptor (Herbert et al.).

Vascular Endothelial Growth Factor

Vascular endothelial growth factor (VEGF) is characterized as a heparin binding protein displaying dual functions, i.e., specifically stimulating the growth of vascular endothelial cells and promoting the permeability of blood vessels or angiogenesis (Neufeld et al.). VEGF functions through two tyrosine kinase receptors, i.e., Flt1 (VEGFR1) and KDR (VEGFR2) that are predominantly expressed on the membrane of vascular endothelial cells (Neufeld et al.; Devries et al.; Terman et al.).

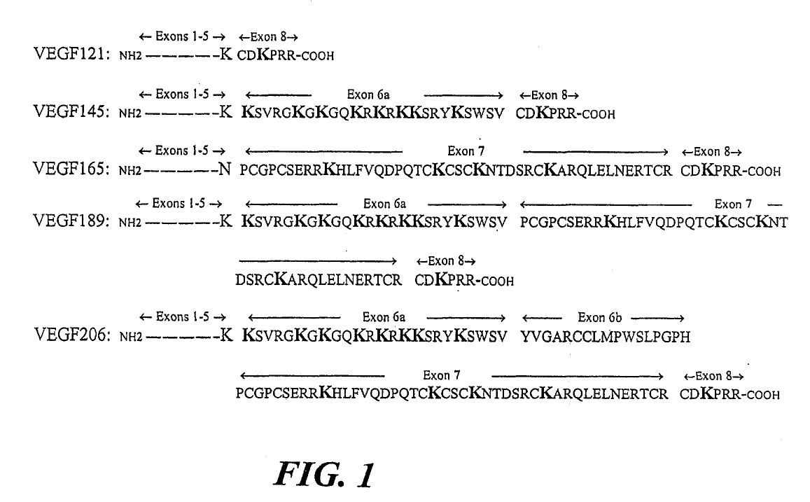

The various molecular forms of VEGF A share a common N-terminal receptor binding domain consisting of 115 amino acids encoded by exons 1-5 and a C-terminal domain containing 6 amino acids encoded by exon 8, together constituting the minimal structure of a functional unit such as VEGF121. Inclusion of the amino acids (i.e., heparin binding domain) encoded by exons 6a, 6b or 7 by alternative splicing leads to the formation of larger molecules, i.e., VEGF145, VEGF165, VEGF189 and VEGF206 (Neufeld et al.; Potorak et al.). Each isoform can form dimers capable of binding to its cognate receptors KDR and/or Flt-1. (Neufeld et al.). Other than VEGF121, which lacks the amino acids encoded by exons 6 and 7, all other isoforms of VEGF A can bind to heparin and HSPG (Park et al.). In addition, VEGF145, VEGF189 and VEGF206 share the same nuclear localization signal encoded by exon 6a (see, FIG. 1)(Zhang et al.).

VEGF165, with 11 lysine residues, is the most abundant form, which has been detected in AIDS-KS lesions (Neufeld et al.; Weindel et al.; Nakamura et al.). Lysine residue 115 existing in other isoforms is replaced by asparagine in VEGF165 (Park et al.). The sequence of 50 amino acids in the C-terminal of VEGF165 is very basic with a pI estimated to be 11.6 (Keck et al.). The inserted 44 amino acids encoded by exon 7 confer on VEGF165 not only a heparin binding ability but also a high affinity for the newly discovered co-receptor Neuropilin-1 expressed in endothelial and tumor cells (Soker et al.). This novel interaction between the product of exon 7 and Neuropilin may explain the higher potency of VEGF165 than VEGF121 in the mitogenic assays (Soker et al.). Since they are located in the region critical for binding to heparin or co-receptor Neuropilin, some lysine (K) residues (a total of 5) in the C-terminal portion of VEGF165 are expected to be exposed on the cell surface and thus chemically accessible. Using human recombinant VEGF165 (R & D Systems, Mckinley Place, Nebr.) as a model, examples contained herein show that lysine residues in this growth factor can be oxidized by LO.

Platelet Derived Growth Factor

Platelet-derived growth factor (PDGF), a polypeptide contained in blood platelets, is a major mitogen in serum for cells of mesenchymal origin and also is a strong chemoattractant for vascular smooth muscle cells (VSMC), fibroblasts, monocytes and neutrophils (Heldin, 1992; Ross et al.; Grotendorst et al.). It is released during the process of blood clotting. PDGF is considered to be a critical growth factor stimulating cell proliferation in embryogenesis, wound healing and tumorigenesis. It may also play a key role in atherosclerosis. Platelets are attracted to and aggregate around collections of fat in the walls of blood vessels (plaques), and the release of PDGF at these sites induces the proliferation and migration of cells in the vessel walls, causing them to narrow. Thus, PDGF also plays an important role in fibroproliferative diseases (Heldin, 1992).

PDGF consists of two different but homologous polypeptides, A and B (˜29-32 kDa) linked by disulphide bonds. Stochastic assembly of the homologous subunits, A chain and B chain, yields the heterodimer PDGF-AB and homodimers PDGF-AA and PDGF-BB. PDGF-AB is a predominant form in human platelets (Heldin, 1992; Johnsson et al., 1982; Hammacher et al.).

Receptor dimerization leads to autophosphorylation of receptors initiating intracellullar signaling for cell migration, proliferation and differentiation (Heldin, 1992; Ullrich et al.). There are two PDGF receptor subunits, α and β, which contain an extracellular ligand-binding domain, a single membrane-spanning region and a cytoplasmic domain with intrinsic protein tyrosine kinase activity. Binding of PDGF ligand at the cell surface induces dimerization of receptors. Since the A chain interacts only with α receptors, while the B chain can bind both α and β receptors, PDGF-AA induces only homodimers of α receptors while PDGF-AB induces α-β receptor heterodimers and β-β receptor homodimers and, PDGF-BB induces all combinations of receptor dimers (Heldin, 1995). Both the mitogenic and chemotactic responses of cells to PDGF require the coordinated action of H₂O₂. It is shown that treatment of VSMC with PDGF-AB elevated intracellular levels of H₂O₂ in these cells even though its source remains undiscovered (Sundaresan et al.).

The human PDGF-A and PDGF-B chains encoded by distinct genes are synthesized as precursors containing signal sequences with a total of 211 and 230 amino acids for the A chain and B chain, respectively (Betsholtz et al.). After propeptide cleavage, a 125 amino acid protein of the PDGF-A chain and a 150 amino acid protein of the PDGF-B chain are generated. A significantly high degree of homology is seen in a region within the mature chains, residues 89-181 of the A-chain is 56% homologous to the B-chain. Both mature chains contain the same basic region VRKKP required for receptor binding (Betsholtz et al.). The lysine-enriched, basic C-terminal peptide (GRPRESGKKRKRKRLKPT) encoded by the exon 6 of the PDGF-A chain was shown to be the matrix binding motif (Raines et al.). PDGF is a basic protein with pI 9.5-10.4 (Heldin et al., 1985). These characteristics of PDGF contribute to its substrate potential for LO.

HIV-1 Tat

In addition to structural and enzymatic proteins, HIV-1 encodes a group of at least six auxiliary regulatory proteins including Tat, a transactivator essential for HIV-1 replication and progression of HIV diseases (Rubartelli et al.). Tat is an RNA-binding protein rather than a DNA-binding protein, which acts predominantly at the level of transcription elongation, rather than initiation (Karn). Transactivation by Tat is entirely dependent upon the presence of and the interaction with the trans-activation responsive (TAR), a 57-nucleotide RNA stem-loop structure (TAR-RNA), that forms at 5′ end of all nascent viral transcripts and located at the region near the start of transcription in the HIV-1 long terminal repeat (LTR) promoter element (Karn; Roy et al.). The basic region of Tat with the R-K (R=arginine, K=lysine) motif mediates the binding to TAR RNA while the active domain with amino acids 1-48 including Cys-rich and core regions of Tat mediates the interaction with cellular transcriptional machinery (Karn). Tat complexes formed with TAR/the positive-transcription elongation factor (P-TEFb)/the RNA polymerase II (RNAPII) increase the transcription from HIV-1 LTR by several hundred-fold higher than that in the absence of Tat (Jeang et al.; Karn). Thus, Tat is an attractive target for anti-HIV-1 therapy.

As a transcriptional activator, Tat is released from HIV-1 infected T cells and then enters endothelial cells where it transactivates inflammatory cytokine genes which, in turn, stimulate production of mitogenic/angiogenic factors, particularly FGF2 and VEGF. This provides a critical microenvironment for angiogenesis. Moreover, extracellular Tat enhances the growth and migration of endothelial cells by binding to and activating cognate membrane receptors via its RGD motif and basic region.

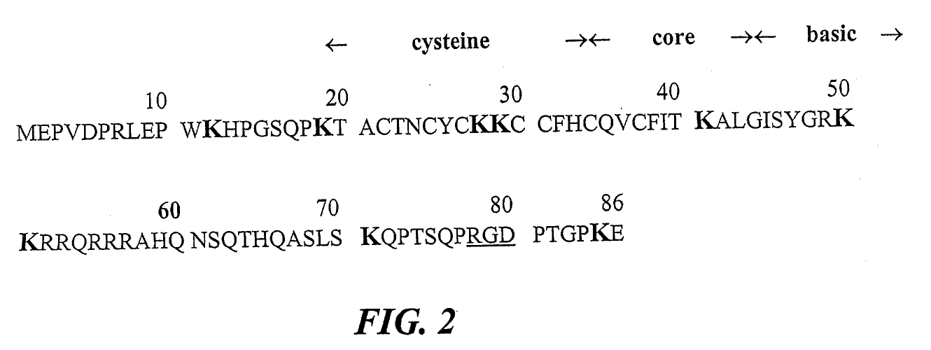

As a basic amino acid, Lysine is critical to the biological functions of Tat. As shown in FIG. 2, the species of Tat with 86 amino acids has a total of nine (9) lysine residues. Point mutations of this Tat by changing K28K29 to EA (K=lysine, E=glutamic acid, A=alanine) in the Cys-rich region, or K41 to T (T=threonine) in the core region, or K50K51 to SG (S=serine, G=glycine) in the basic region totally abolished or significantly reduced its transcriptional activity (Jeang et al.).

Tat is a basic protein with a pI of 10.04 (Albini et al., 1996), containing 86-104 amino acids (10 kDa) encoded by two exons (FIG. 2) (Bieniasz et al.). The first 72 residues are encoded by the first exon and composed of several functional regions. The cysteine (Cys)-rich region (amino acids 22-37) is essential for transactivation (Garcia et al.; Ruben et al.) and mediates the formation of metal-linked dimers in vitro (Frankel et al.). The basic region confers RNA binding properties to Tat and is also involved in Tat nuclear localization and uptake by the cell (Endo et al.; Calnan et al.; Chang et al., 1995). The core region includes a common sequence FXXKXLGI (X: other amino acids) found in HIV-1, HIV-2 and SIV Tat (Bieniasz et al.). Amino acids 1-48 together circumscribe a minimal activation domain for the Tat protein (Carroll et al.; Derse et al.). The remaining C-terminal residues are encoded by the second exon and contain an RGD motif. Although this region is not required for transactivation, it is crucial in binding to integrin receptors (Barillari et al., 1993).

Implication of Growth Factors in Tumorigenesis

FGF2, PDGF and VEGF, key growth factors for a variety of cells, stimulate mitogenesis and angiogenesis in the development of tumors and AIDS-associated malignancies (Neufeld et al.; Heldin, 1992; Basilico et al.).

FGF2 has been established as having dual roles in both mitogenesis and angiogenesis on the development of tumors. Overexpression of intracellular FGF2 has been demonstrated to induce autocrine transformation of NIH 3T3 cells (Yayon et al.). A recent comprehensive analysis of FGF2 and FGF receptors (FGFR) in 60 human tumor cell lines with a wide range of tissue origins such as the breast, colon, renal, prostate, ovarian, lung and brain, etc., shows that FGFR and bFGF were expressed in 90% and 32%, respectively, of tested cell lines (Chandler et al.). Of the 103 breast cancer samples, FGF2 transcripts were positive in 96 cases (95%) (Penault-Llorca et al.). These results provide strong evidence that FGF2 signaling pathways are active in a majority of human tumors.

Studies have isolated FGF2 from tumors as being one of the first angiogenic factors required to grow in mass (Folkman et al.). Patients with non-small cell lung carcinomas that exhibited high levels of FGFR1 expression showed significantly shorter survival times (Volm et al.). Thus, high levels of FGF2 in tumors are considered to be a poor prognostic determinant (Wellstein et al.). Upregulation of FGF2 in malignant melanomas together with a high mitogenic sensitivity of normal melanocytes to FGF2 has led to a hypothesis that FGF2 may be an oncogene whose activation is important in the etiology of human melanomas (Basilico et al.; Halaban et al.). Antisense oligodeoxynucleotides against human FGF2 inhibited the proliferation of melanoma cells and their ability to form colonies in soft-agar medium (Becker et al.).

The angiogenic process of FGF2 is regulated by a FGF2 binding protein (FGF2-BP). FGF2-BP is secreted by tumor cells and serves as an angiogenic switch molecule. FGF2-BP binds to inactive FGF2 immobilized by HSPG in the ECM resulting in the release of soluble and bioactive form of FGF2, which stimulates vascular endothelial cells and SMC for angiogenesis (Czubayco et al.).

High levels of VEGF were also detected in various types of tumors occurring in the lung, breast, uterine cervix, prostate, etc., indicating its major role in tumorigenesis and metastasis (Neufeld et al.; Ambs et al.; Kranz et al.; Fujimoto et al.; Borgstrom et al.). Notably, VEGF expression is upregulated by hypoxia. Potentiation of VEGF production in hypoxic areas of solid tumors significantly contributes to VEGF-driven tumor angiogenesis (Shweiki et al.).

Recent studies involving the PDGF-B autocrine system have been carried out in a number of human tumors including prostate carcinoma, melanoma, soft tissue tumors, basal cell cancer and leukemias, etc. (Potapova et al.). Over 150 human tumor lines, representing 26 tumor types, express one or both PDGF-A and PDGF-B chains and 55 human tumor lines also express one or both PDGF α and β receptors (Mercola). In one series of analysis including 42 biopsies of human gliomas, all tumors exhibited PDGF-B at higher levels than normal nervous tissues and 83% expressed the PDGF-β receptor at up to 30-fold higher levels than other low-grade neural tumors and benign gliomas (Mauro et al.). Morphological studies of human glioblastomas further demonstrated that two PDGF receptors were expressed in different patterns; the α-receptor was mainly distributed in the tumor cells whereas the β-receptor was generally located in the supporting stoma. Although both PDGF-A and -B chains were found in these brain tumors, the high level of the β-receptor in the stroma was often seen in more malignant tumors than in benign or less malignant tumors (Hermansson et al.). A similar malignancy-dependent expression of PDGF and PDGF receptors was also determined in fibrosarcomas (Smits et al.).

DNA sequence analysis has revealed that nucleotide sequences of five regions of the human c-sis gene are homologous to sequences of the transforming region (v-sis) of simian sarcoma virus (SSV). Amino acid sequence analysis of the PDGF-B chain shows identity to the amino acid sequence predicted from the c-sis sequence over 109 amino acid residues, thus establishing that the c-sis, a human protooncogene, encodes a polypeptide precursor of the PDGF-B chain (Johnsson et al.). These findings provide a link between growth factors and oncogene products and point out a mechanism whereby oncogene products transform cells, i.e., by subversion of the mitogenic pathway of growth factors (Heldin, 1992). The PDGF-B, a c-sis gene product, is the first cellular growth factor shown to correspond to a known viral oncogene (PDGF-B/v-sis), suggesting its potential role in tumorigenesis (Heldin, 1992; Potapova et al.). As illustrated by DNA transfection assays, normal PDGF-B chain gene has transforming properties, as does the A chain gene albeit with less potency (Heldin, 1992). Clonal cell lines with stable expression of PDGF-B/v-sis derived from benign, nontumorigenic glioblastoma cells exhibited a high degree of tumorigenic and metastatic phenotype (128). It has been recognized that the PDGF-A chain and the PDGF-B chain/c-sis was continuously expressed in some human tumors, e.g., osteosarcoma (Heldin et al., 1980) and fibrosacoma (Eva et al.), consistent with an autocrine and/or paracrine mechanism regulating tumor growth. For example, the mitogenic activity of media conditioned by U-2 OS, an osteosarcoma cell line, was abolished by PDGF antibodies (Betsholtz et al.).

Our studies have shown that FGF2, VEGF165 and PDGF-AB are substrates of LO and their mitogenic activities were markedly reduced following LO oxidation. In light of critical roles of FGF2, VEGF and PDGF in cell transformation, tumorigenesis and tumor angiogenesis, we hypothesize that LO may be an effective antagonist to prevent the development of tumors that depend on autocrine and/or paracrine mechanisms activated by FGF2, VEGF and/or PDGF.

Pathological Agents in Tumorigenesis of AIDS Associated Kaposi's Sarcoma (AIDS-KS)

Among all pathogenic agents, Tat of HIV-1, FGF2 and VEGF are most important for the development of AIDS-KS (Ensoli et al., 1998). Tat can be released from HIV-1 infected cells and enter other cells, where it translocates to the nucleus in an active form (Ensoli et al., 1993). In HIV-1 uninfected cells, Tat can transactivate cellular genes of inflammatory cytokines, which induce the production of growth factors, angiogenic molecules and chemokines (Buonaguro et al.; Scala et al.). Tat harbors a critical microenvironment for etiologic factors such as human herpesvirus 8 (HHV-8) to initiate the early hyperplastic-proliferative lesions and/or to facilitate the progression of late malignancy.

Tat increased HHV-8 viral loading in KS (Harrington et al.) and stimulated expression of cellular homolog genes of HHV-8 such as vGCR which can induce cell transformation (Yen-Moore et al.). Tat, but not HIV-1, is detectable in AIDS-KS lesions (Ensoli et al., 1994, Nature; Bovi et al.) and is, at least in part, implicated in the aggressiveness of this tumor. Extracellular Tat enhances the growth, migration and invasion of AIDS-KS cells by binding to the target cell membrane and activating signal molecules such as p125 FAK, paxillin, p130, MAP kinases, etc. (Ganju et al.). The RGD (arginine-glycine-aspartic acid) motif of Tat recognizes integrins, i.e., α5 μl and αvβ3 while its basic region has a high affinity for the VEGF receptor 2 (KDR1). α5 μl, αvβ3 and KDR1 are all expressed in AIDS-KS (Barillari et al.; Brown et al.).

The crucial role of Tat in KS pathogenesis is further demonstrated in studies of Tat-expressing, transgenic mice, which developed hyperplasia of the dermis followed by the appearance of vascular tumors. Tumorigenesis is seen only in male mice (Vogel et al.). Tat has potent angiogenic activity as evidenced by inducing proliferation of cytokine-stimulated endothelial cells and stabilizing the capillary-like network formed by endothelial cells on a matrix support (Albini et al., 1995). The angiogenic activity of Tat in vivo is strongly potentiated by heparin although high concentrations of heparin inhibit Tat activity (Albini et al., 1994). Notably, Tat competes with FGF2 for matrix binding and helps maintain this growth factor in a soluble form enhancing its biological activity.

As indicated in animal studies, inoculation of Tat in the presence of a suboptimal dose (no lesion produced alone) of FGF2 induced synergistic effects on the number of mice developing lesions and the intensity of histological alterations including the spindle cell production and proliferation and angiogenesis in nude mice (Ensoli et al., 1994, Nature; Albini et al., 1994). In addition, Tat can activate the expression of bcl-2, a protooncogene known to prolong survival of quiescent non-proliferative cells by inhibiting the process of apoptosis (Zauli et al.). Immunohistochemical analysis shows that AIDS-KS lesions contain significant levels of bcl-2 distributed in endothelial and spindle cells of lesions, particularly those in the nodular late-stage of AIDS-KS (Bohan-Morris et al.). The presence of detectable extracellular Tat in sera from AIDS patients (97) and in AIDS-KS lesions (Ensoli et al., 1994, Nature) strongly supports its role as a critical progressive factor for AIDS-KS. Thus, inactivation of key biological functions of Tat has considerable prophylactic and therapeutic value in the control of AIDS-KS.

FGF2 is also a key mediator in the development of AIDS-KS. This growth factor is highly expressed in AIDS-KS primary lesions at the mRNA and protein levels (Ensoli et al., 1994, Nature; Xerri et al.). In fact, inoculation of FGF2 in nude mice resulted in the formation of KS-like lesions (Ensoli et al., 1994, Nature). The inhibition studies with specific neutralizing antibodies or antisense oligodeoxynucleotides directed against FGF2 mRNA have shown that FGF2 is required for the production of KS lesions by inoculation of AIDS-KS cells in nude mice (Ensoli et al., 1989; Ensoli et al., 1994, J. Clin. Invest.). Cultured AIDS-KS cells activate autocrine and/or paracrine mechanisms of FGF2 enhancing the growth and angiogenesis of endothelial cells in vitro and in vivo (Ensoli et al., 1989; Samaniego et al.). As discussed, Tat of HIV-1 and inflammatory cytokines stimulate the production of FGF2 in AIDS-KS cells and in normal endothelial cells in a synergistic fashion (Ensoli et al., 1994, Nature; Albini et al., 1994; Samaniego et al., 1995; Samaniego et al., 1997; Samaniego et al., 1998). Inflammatory cytokine-activated endothelial cells produce FGF2 and induce KS-like lesions in nude mice (Samaniego et al., 1997). Tat with inflammatory cytokines promotes the development of angioproliferative KS-like lesions in nude mice via production of FGF2 and expression of αvβ3 integrin that is elicited by this growth factor and binds to the RGD region of Tat (Barillari et al., 1999). Thus, auto- and paracrine actions of FGF2 mediate AIDS-KS cell growth and represent an important therapeutic target for AIDS-KS intervention. TNP-470, an analog of fumagillin that was first isolated from Aspergillus Fumagatus fresensium, inhibits FGF2-induced endothelial cell division and has been used in clinical trial for KS (Dezube et al.).

Two principal features of AIDS-KS, i.e., aberrant growth of vascular structures with overproliferation of spindle KS cells derived from endothelial cells and enhanced vascular permeability (http://cancernet.nch.nih.gov/cgi-bin/srchcgi.exe; Antman et al.), strongly support the notion that VEGF is another key mediator in addition to Tat and FGF2 in the pathogenesis of this malignancy associated with AIDS. As illustrated, AIDS-KS tissues and cells in primary culture expressed high levels of VEGF and VEGF receptors (Brown et al.; Massod et al.). The predominant form of VEGF mRNA in KS cells is the 3.9 kb transcript that encodes the VEGF165 with 165 amino acids (Weindel et al.). A further study on the VEGF expression in various AIDS-KS lesions such as the pleural effusion, lung, oral mucosa and skin isolated from a number of patients showed that primary cultures of AIDS-KS cells released large amounts of VEGF to the conditioned media reaching 12.1-21.4 ng/ml. Combination of both anti-VEGF and anti-FGF2 antibodies completely depressed the growth of endothelial cells stimulated with such AIDS-KS cells-conditioned media while stimulative activities partially remained in the presence of only one of the two antibodies. The molecular weight range of VEGF monomers shown in this study was similar to that of recombinant VEGF165 as determined by Western blotting (Nakamura et al.). Thus, VEGF165 is the major form expressed in AIDS-KS although other growth factors showing sequence homology to VEGF such as VEGF B, C and D and placental growth factor were all detected in this tumor (Mihalcea et al.; Masood et al.). VEGF receptors Flt-1 and KDR appear to play an important role in VEGF signal transduction implicated in the development and progression of AIDS-KS. A mutant VEGF incapable of activating KDR receptors produced no proliferative response (Marchio et al.). Targeting VEGF receptors with the fused VEGF165 or VEGF121 to diphtheria toxin effectively suppressed proliferation of endothelial cells and KS cells but not VSMC (Arora et al.). Both VEGF and FGF2 are induced by inflammatory cytokines in AIDS-KS and synergistically promote cell growth and vascular permeability (Samaniego et al., 1998). In briefly, a large body of evidence shows that VEGF represents another key therapeutic target for AIDS-KS directly involving inhibition of tumor angiogenesis, a basic concept and strategy for tumor control (Krown).

By comparing the chemical features and biological functions of Tat, FGF2 and VEGF, we have come to the following conclusions: 1) They are all basic proteins with pI>9.0; 2) They all have a lysine and arginine-rich basic domain in their C-terminals (FIG. 3); 3) They are all distributed in the ECM, and exhibit a high affinity for heparin and HSPG; 4) They all can be internalized by the cell and localized in the nuclei; 5) They all target endothelial cells, stimulate their proliferation and induce angiogenesis; and more importantly, 6) They all can compete and/or synergize with each other to exert their biological functions. This all supports the credibility of our invention on the development of novel therapeutic agents that directly target the key pathogenic factors such as Tat, FGF2 and VEGF would be an effective strategy for the prevention and therapy of AIDS-KS.

### EXAMPLES

The following examples are presented to illustrate the advantages of the present invention and to assist one of ordinary skill in making and using the same. These examples are not intended in any way otherwise to limit the scope of the disclosure.

### Example I

### Growth Factors and Angiogenic Factors as Substrates of Lysyl Oxidase

FGF as a Substrate of LO

The following experiments were carried to demonstrate that FGF2 is a substrate of LO. The oxidation of a primary amine substrate by LO yields stoichiometric amounts of the corresponding aldehyde, hydrogen peroxide and ammonia, as shown: RCH₂NH₂+O₂+H₂O→RCHO+H₂O₂+NH₃ (Kagan, 1986). To assess the substrate potential of FGF2, LO-dependent H₂O₂ release was monitored in assays using FGF2 as the sole substrate, using both the optimum temperature for LO activity of 55° C. and the physiological temperature of 37° C. (Trackman et al., 1981).

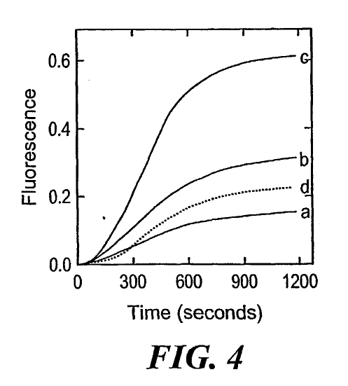

LO reaction with FGF2 as a substrate was assessed by a horseradish peroxidase-coupled fluorescence assay for H₂O₂ production. Reaction mixtures contained 0.25 mg of sodium homovanillate(HVA), 40 μg of HRP, and FGF2 at doses as indicated below in 0.05 M sodium borate buffer, pH 8.2, in a final volume of 2 ml. Assays were initiated by the addition of LO to reaction mixtures. H₂O₂ release was continuously monitored at excitation and emission wavelengths of 315 and 425 nm, respectively, at a constant temperature of 55° C. or 37° C., as specified, in the thermostatted cuvette chamber of an SLM Aminco Bowman Series 2 Luminescence Spectrometer. All activity data were corrected for background rates of H₂O₂ release determined in complete assay mixtures supplemented with 200 μM BAPN, an irreversible inhibitor of LO. In FIG. 4, solid curves represent the time course of H₂O₂ release at 55° C. in the reaction of 0.16 μM LO with 0.23 (a), 0.45 (b) and 0.9 (c) μM FGF2, respectively. Dotted curve (d) represents the time course of H₂O₂ release in the reaction of 0.16 μM LO with 0.9 μM FGF2 at 37° C. The same data were obtained from three separate experiments.

As shown in FIG. 4, incubation of 0.16 μM LO with 0.23, 0.45 or 0.9 μM concentrations of 18 kDa human recombinant FGF2 resulted in rates of H₂O₂ release with increasing concentrations of FGF2 (solid curves a, b and c). The curves exhibited an initial lag phase before the maximum rate continued to a plateau. The plateau evidences the complete release of H₂O₂ seen in each curve. Introduction of additional aliquots of functional LO within the plateau phase of the curves did not cause additional H₂O₂ release, which indicated that the plateaus represent maximum degrees of lysine oxidation in FGF2.

The amount of H₂O₂ released at the plateau regions was calculated by reference to a standard curve that related fluorescence units to moles of H₂O₂ produced. The molar ratios H₂O₂ released per mole of FGF2 present at the plateau regions were 14.1, 14.6 and 14.2 for LO reacted with 0.23, 0.45 and 0.9 μM FGF2, respectively, yielding a mean±standard deviation of 14.3±0.3, consistent with the 14 lysine residues in each native molecule of FGF2.

The assays at the physiological temperature of 37° C. with 0.9 μM FGF2 (dotted curve d) yielded a maximum of 5.3 moles of H₂O₂ per mole of FGF2 compared to the molar ratio of 14.2 moles (H2O₂/FGF2) obtained at 55° C. from the same concentration of the growth factor (compare curves c and d). These results indicate that conformational changes may have allowed for all of the lysine residues of FGF2 to be oxidized by LO at 55° C. A limiting number of lysines oxidized at 37° C. is presumed to reflect sufficient retention of the native structure to permit only the normally exposed surface lysines to be oxidized by LO under these more physiological conditions.

To determine crosslinking of FGF2 monomers as a result of its oxidation by LO, human recombinant FGF2 (18 kDa) was iodinated and ¹²⁵1-labeled FGF2 (5 μg) and was then incubated with 4 μg of LO in 0.1 M sodium borate buffer, pH 8.2 in a final volume of 100 μl, at 37° C. for 1 h. The reaction was quenched by the addition of 100 μM BAPN followed by incubation at 37° C. for 30 min. Aliquots of the incubated reaction mixtures were analyzed by SDS-PAGE (15%) and autoradiography. Lane 1 represents [¹²⁵]FGF2; lane 2 represents [¹²⁵1]FGF2+LO. The relative electrophoretic mobilities of pre-stained molecular weight standards are indicated under MW.

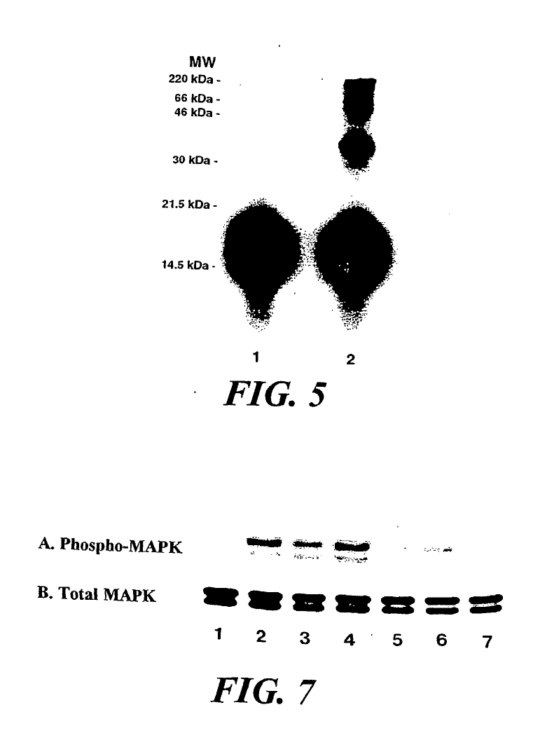

As shown in FIG. 5, incubation of FGF2 with LO induced the appearance of covalent dimers, oligomers and higher polymers (lane 2) derived from the 18 kDa monomers (lane 1). The diner of FGF2 is a prominent product of the growth factor oxidized by LO. Thus, FGF2 is found to be a productive substrate for LO and to form crosslinked polymers following the oxidation of peptidyl lysine by LO.

TABLE 1
Oxidation of Other FGF by LO in vitro

| | | | Oxidized Lysines | Oxidized Lysines |
|---|---|---|---|---|
| Growth | Total | Total | per Molecule | per Molecule |
| factors | AA | Lysines | 55° C. | 37° C. |
| FGF4 | 206 | 15 | 14.5 | 3.6 |
| FGF5 | 267 | 21 | 18.0 | 10.0 |
| FGF6 | 198 | 7 | 7.2 | 3.1 |

Using the fluorometric assay (Trackman et al., 1981) to monitor LO-dependent H₂O₂ release in vitro, the substrate potential of other growth factors was also assessed. Major members of the FGF family tested included FGF 1, 4, 5, 6, 7 and EGF (note: All factors are human recombinant forms purchased from R & D Systems, Inc., Minneapolis, Minn.). FGF4, FGF5 and FGF6 were found to be substrates of LO, while FGF1, FGF7 and EGF were not.

For the substrate polypeptides as shown in Table 1, almost all lysine residues in these proteins were oxidized by LO at the optimum temperature of 55° C. whereas only limited peptidyl lysines reacted with LO at the physiological temperature of 37° C. The molar ratios of H₂O₂ released per molecule of substrates upon LO action at the physiological temperature were 3.6, 10.0 and 3.1 for FGF4, FGF5 and FGF6, respectively. These results may reflect the fact that only those lysines on the surface of molecules are chemically accessible and oxidized by LO at the physiological temperature. FGF4 (the alternative name: K-FGF/HST-1), was discovered by screening for genes present in human stomach tumors or Kaposi's sarcoma (Basilico et al.). High levels of FGF4 were detected in embryo carcinoma, teratocarcinoma and germ cell tumors. FGF4, FGF5 and FGF6 all are oncogenes expressed in embryo tissues and all capable of inducing cell transformation (Basilico et al.).

VEGF as a Substrate of LO

A human recombinant disulfide-linked dimer of VEGF165 (MW: 42 kDa)(R & D Systems) as a sole substrate was tested by using the standard in vitro LO catalysis assays as described above for FIG. 4 (Trackman et al., 1981). Curves represent the time course of H₂O₂ release at 55° C. (a) or 37° C. (b) in the reaction of 0.10 μM LO with 0.45 μM VEGF165. All activity data were corrected for background rates of H₂O₂ release determined in complete assay mixtures supplemented with the same amounts of LO preincubated with 100 μM BAPN for 15 min at 37° C. The same data were obtained from three separate experiments.

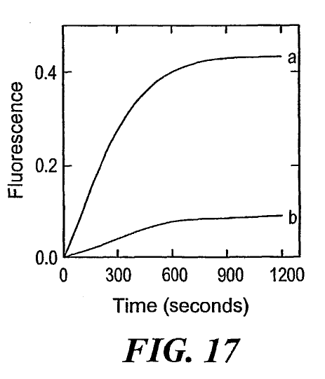

As shown in FIG. 17, results indicated that incubation of 0.1 μM LO with 0.45 μM VEGF165 released 20 moles of H₂O₂ per mole of VEGF165 at 55° C. and 4 moles of H₂O₂ per mole of VEGF165 at 37° C. were released at the plateau regions of reaction curves. The maximum oxidation of substrates by LO was performed at the optimal temperature of 55° C. These results indicated that almost all lysine residues per molecule of the VEGF165 dimer (containing a total of 22 lysine residues) were oxidized by LO at 55° C. while 4 lysine residues per molecule of the VEGF165 dimer were oxidized by LO at 37° C. Thus, VEGF has been determined to be a substrate of LO.

PDGF as a Substrate of LO

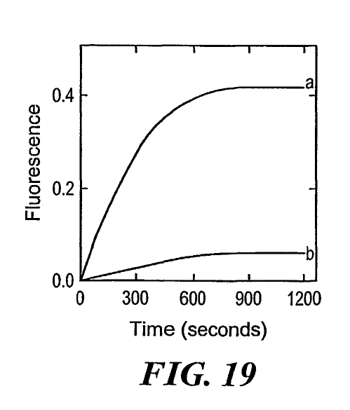

The standard in vitro LO catalysis assays as described above for FIG. 4 (Trackman et al., 1981) using human recombinant PDGF-AB (R & D Systems) as a sole substrate was tested. The mature A chain (residues 87-211) contains 14 lysine residues while the mature B chain (residues 81-190) possesses 7 lysine residues. In FIG. 19, curves represent the time course of H₂O₂ release at 55° C. (a) and 37° C. (b) in the reaction of 0.11 μM LO with 0.46 μM PDGF-AB, respectively. All activity data were corrected for background rates of H₂O₂ release determined in complete assay mixtures supplemented with the same amounts of LO preincubated with 100 μM BAPN for 15 minutes at 37° C. The same data were obtained from three separate experiments.

As shown in FIG. 19, incubation of 0.11 μM LO with 0.46 μM 27 kDa human PDGF-AB resulted in the release of H₂O₂ probed by the fluorescent acceptor HVA in the presence of HRP (Trackman et al., 1981). Results indicate that there was a rapid release of H₂O₂ followed by a plateau, which indicates complete oxidation of lysine residues. The molar ratios of H₂O₂ released per mole of PDGF-AB at the plateau regions were 19.5 and 3.0 at assay temperatures of 55° C. (curve a) and 37° C. (curve b), respectively. The molar ratios were determined by reference to a standard curve relating fluorescence units to moles of H₂O₂ produced in this assay system. These results indicated that nearly all of the 21 lysine residues of the PDGF-AB substrate were oxidized by LO at 55° C., but only small number of lysine residues was oxidized at 37° C. This may be explained by the limited surface exposure in the native structure of the lysine residues to permit access to and catalysis by LO under the more physiological condition at 37° C.

Tat as a Substrate of LO

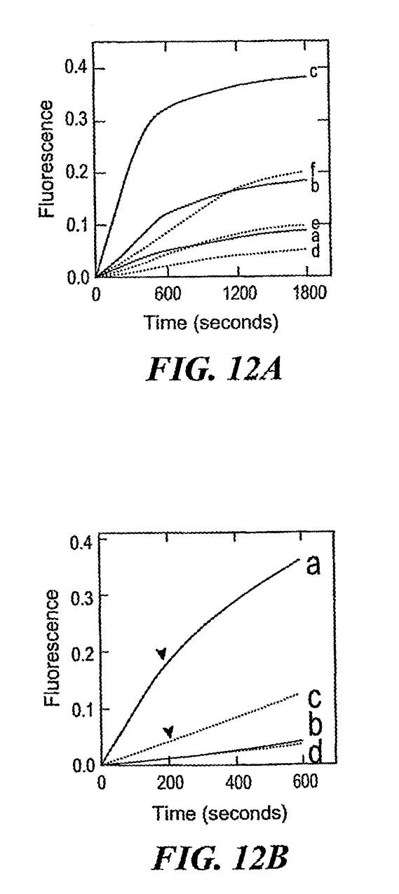

To analyze the substrate potential of Tat, the standard in vitro LO catalysis assays as described in FIG. 4 (Trackman et al., 1981) was performed at the optimum temperature of 55° C. and at the physiological temperature of 37° C. A recombinant Tat with 9 lysines in a total of 86 amino acids was used as a sole substrate. FIG. 12A represents a dose and temperature-dependent response. Curves represent the time course of H₂O₂ release at 55° C. (solid) or 37° C. (dotted) in the reaction of 0.10 μM LO with 0.22 (a and d), 0.43 (b and e) and 0.85 (c and f) μM Tat, respectively. All activity data were corrected for background rates of H₂O₂ release determined in complete assay mixtures supplemented with the same amounts of LO preincubated with 100 μM BAPN for 15 min at 37° C. FIG. 12B represents the effects of BAPN on the reaction of LO with Tat. LO at 0.10 μM was mixed with 0.85 μM Tat at 55° C. (solid curves) or 37° C. (dotted curves). At 200 sec after the reaction start (curves a and c), 100 μM BAPN was added to mixtures (arrow heads). Reactions were allowed to continue for an additional 400 sec. LO at the same concentration preincubated with 100 μM BAPN for 15 min at 37° C., then reacted with the same amount of Tat under same conditions was assayed as negative control (curves b and d). The same data were obtained from three separate experiments.

As shown in FIG. 12A, incubation of 0.1 μM LO with 0.22 (curve a), 0.43 (curve b) and 0.85 (curve c) μM Tat, respectively, at 55° C. for 30 min (solid curves) resulted in release of H₂O₂ probed by the fluorescent dye HVA in the presence of HRP (136). The results indicated a rapid H₂O₂ release followed by a plateau phase showing the complete release of H₂O₂. The molar ratios of H₂O₂ released per mole of Tat at 0.22 (curve a), 0.43 (curve b) and 0.85 (curve c) μM at the plateau regions are 8.42, 8.88 and 9.31, respectively, yielding a mean±standard deviation of 8.87±0.45 at 55° C. The molar ratios were determined by reference to a standard curve relating fluorescence units to moles of H₂O₂ produced in this assay system.

At the physiological temperature of 37° C. (dotted curves), the LO catalysis assay resulted in a maximum release of 4.85, 4.75 and 4.80 (average 4.80±0.05) moles of H₂O₂ per mole of Tat, using 0.1 μM LO with 0.22 (curve d), 0.43 (curve e) and 0.85 (curve f) μM Tat, respectively. FIG. 12A also shows time course studies in which, for example, 0.22 (curve d), 0.43 (curve e) and 0.85 (curve f) μM Tat yielded 2.12±0.05, 4.05±0.05 and 4.80±0.04 moles of H₂O₂ released per mole of Tat at 10, 20 and 30 min, respectively, after incubation with 0.1 μM LO at 37° C.

These results indicate that nearly all of the 9 lysine residues of the Tat substrate were oxidized by LO at 55° C., but only a small number of lysine residues was oxidized initially under the physiological temperature condition. The lysine residues may have limited exposure in the native structure of Tat to permit access to and catalysis by LO. Notably, at least two lysine residues are very easily accessible since 2.12 moles of H₂O₂ per mole of Tat were released during the first 10 minutes of reaction at 37° C.

To further distinguish Tat's substrate potential of LO, LO was preincubated with a potent active site-directed inhibitor of LO, BAPN. LO was preincubated with 100 μM BAPN for 5 minutes, which totally inhibited its reaction with Tat (FIG. 12B, curves b at 55° C. and d at 37° C.). However, after the reaction of LO with Tat was initiated, the addition of the same amount of BAPN to the mixture, for example, at the time point of 200 seconds, failed to block H₂O₂ release (curves a at 55° C. and c at 37° C.). This suggested a higher affinity of LO with Tat than with BAPN. These results led to the conclusion that Tat is an excellent substrate of LO.

### Example II

### Anti-Mitogenic/Angiogenic and Anti-Tumorigenic Effect of Lysyl Oxidase

Tumorigenesis of cells may result from a constitutive interaction of growth factors with their corresponding receptors (Sporn et al.). Autocrine transformation can occur under conditions where growth factors are either secreted and bind to cell surface receptors to form an external autocrine loop, or they are not secreted, but directly target their intracellular receptors to form an internal autocrine loop (Browder et al., 1989, Cancer Cell). Recent observations suggest that internal autocrine stimulation is a major mode of malignant transformation (Keating et la.; Browder, 1989, Mol. Cell. Biol.).

For example, Kaposi's FGF (i.e., FGF4) oncogene and Simian sarcoma virus oncogene, v-sis (encoding a homologue of the PDGF B chain) induce cell transformation as a result of the activation of an internal autocrine loop (Ensoli et al., 1989; Keating et al.; Northfelt et al.). We have assessed the effects of LO on tumor cell growth by using highly transformed NIH 3T3 6-1 cells as a model. These cells are oncogenic because they over-express FGF2, which then activates an internal autocrine mechanism stimulating cell proliferation. These cells have an enhanced proliferation rate, are not density arrested, and are capable of anchorage-independent growth (Yayon et al.; Rogelj et al.). Like other types of internal autocrine transformed cells, e.g., those induced by Kaposi's FGF and v-sis oncongenes (Ensoli, 1989; Keating et al.; Northfelt et al.), they poorly express FGF receptors on the cell surface. Thus, the FGF2 produced occurs within the cell and is not present in the conditioned medium. Animal studies showed that inoculation of NIH 3T3 6-1 cells to syngeneic NIH/NSF mice induced rapidly growing tumors within a week. Such tumors can be cultured in vitro and shown to produce substantial biologically active bFGF (Yayon et al.; Rogelj et al.).

LO Inhibition of Tumorigenesis in FGF2 Transformed Cells

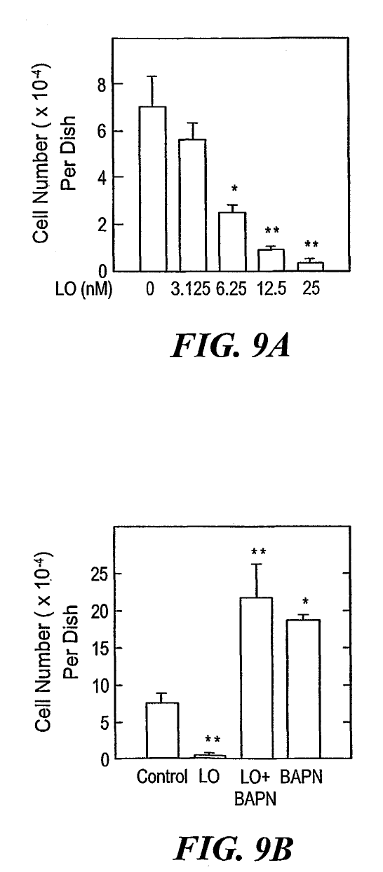

Transformed NIH 3T3 6-1 cells (2×10⁴) overexpressing FGF2 were plated and incubated overnight in 35 mm-dishes containing 2 ml 10% fetal bovine serum (FBS)/Dulbecco's Modified Eagle's (DME) medium, and then refed with 0.3% FBS/DMEM with different treatment regimens as shown in FIGS. 9A and 9B. FIG. 9A represents a dose response. Cells were treated with LO at various doses for a total of 6 days. Cultures were refed with fresh media supplemented with LO, as indicated, every two days. After treatment, cells were harvested by trypsinization and counted with a hemacytometer. FIG. 9B represents the effects of inactivation of LO by BAPN on tumor cell growth. LO was present at 25 nM and BAPN at 200 μM as indicated. Data represent the means (±sd) of three separate experiments each assessed with triplicate dishes. Asterisks indicate values significantly different from the control as determined by ANOVA analysis: * p<0.05, ** p<0.01.

The phenotype of the proliferated NIH 3T3 6-1 cells under serum-starvation conditions was examined. The cells had multiplied actively for more than 5 days under these conditions consistent with their transformed phenotype. In contrast, the parental cells all died within 5 days of culture under the same conditions. These results established the condition to assay LO effects on tumor cell growth by using 0.3% FBS/DMEM as a standard.

Exposure of cells to LO at nM concentration range for 6 days resulted in the dose-dependent inhibition of tumor cell proliferation, as shown in FIG. 9A. Maximal inhibition was obtained at 25 nM LO which decreased the cell number by 95% compared to the control. The LO-mediated growth inhibition of these cells was abolished by the addition of 200 μM BAPN in the medium, amounting to 3-fold proliferation than the control while BAPN in the absence of added LO enhanced tumor cell growth by 2.5 fold than the control, shown in FIG. 9B.

Numerical and morphological alterations in tumor cells treated with LO were observed under phase-contrast microscopy (25×). FIG. 10 comprises the following: A, control; B, 25 nM LO; C, 25 nM LO+200 μM BAPN; D, 200 μM BAPN alone; and E and F, LO uptake by NIH3T3 6-1 cells. In E and F, cells grown on coverslips were exposed to FITC-conjugated LO (0.16 μg/ml) for 2 h, then fixed and examined by fluorescent microscopy (100×).

As shown in FIG. 10, in sharp contrast, LO treatment induced cell death as evidenced by a number of non-adherent cells. A few attached cells exhibited swollen bodies with several longer processes (compare FIGS. 10B with 10A). The presence of BAPN in the culture medium to inhibit either exogenously added LO (FIG. 10C) or endogenous LO (FIG. 10D) significantly promoted cell replication (compare FIGS. 10C and 10D with 10A). These results strongly indicated that catalytically active LO inhibits proliferation of these tumor cells. This inhibition requires the preservation of LO activity since BAPN, an active site inhibitor of LO, blocked its inhibition of transformed cell growth. As noted, the growth of transformed NIH 3T3 6-1 cells is fully dependent on the activation of an internal autocrine signal transduction loop by FGF2 endogenously produced in excess by these cells (Yayon et al.; Rogelj et al.). Since FGF2 is a substrate of LO, FGF2 oxidation by LO should lead to disruption of the internal autocrine loop inducing inhibition of growth and final death of tumor cells. Excess growth seen in the presence of BAPN most likely reflects the inhibition of endogenous LO activity, which may intrinsically regulate intracellular FGF2 action. Indeed, direct assays of aliquots of medium conditioned by NIH 3T3 6-1 cells for 16 h in 0.3% serum revealed the presence of detectable levels of LO activity (2798±562 cpm ³H₂O per h per 10⁶ cells). LO is also internalized by these cells determined by using FITC-conjugated LO as a marker (FIGS. 10E and 10F). Thus, it is likely that the internalized LO oxidatively modifies the FGF2 endogenously produced within these cells.

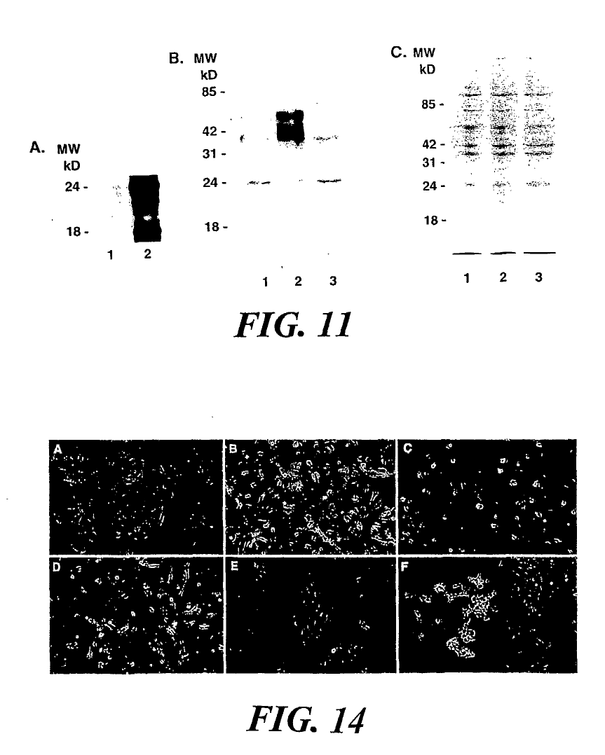

The possibility that exposure of cells to LO alters the physical state of intracellular FGF2 was assessed by Western blotting with monoclonal anti-FGF2 of cell lysates. Subconfluent NIH 3T3 and NIH 3T3 6-1 cells were incubated for 20 h in 0.3% FBS/DMEM with or without additives as indicated below. Cells were suspended by trypsin treatment and lysed. Aliquots of lysates containing 50 μg of cell protein were analyzed by Western blotting of SDS-PAGE electrophoretograms probing with monoclonal anti-FGF2 (FIGS. 11A and 11B). Total protein was visualized by Coomassie blue staining of SDS-PAGE electrophoretograms (FIG. 11C). FIG. 11A represents FGF2 expression in NIH 3T3 (lane 1) and NIH 3T3 6-1 cells (lane 2). FIG. 11B represents forms of intracellular FGF2 in NIH 3T3 6-1 cells incubated in the absence (lane 1) or presence of 25 nM LO (lane 2) or presence of 25 nM LO which had been preincubated with 100 μM BAPN for 15 min at 37° C. (lane 3). FIG. 11C represents the total protein profiles in control and LO-treated cells with treatments in each lane corresponding to those in FIG. 11B.

As shown (FIG. 11A), comparison of FGF2 content of the parental NIH 3T3 and transformed NIH 3T3 6-1 cells reveals significantly increased levels of 18 to 24 kDa forms of FGF2 in NIH 3T3 6-1 cells, as expected. Exposure of the NIH 3T3 6-1 cells to 25 nM LO induced the formation of crosslinked dimers and oligomers of FGF2 within the cells (FIG. 11B, lane 2). Oligomer formation was prevented by inhibiting LO by preincubation with BAPN (FIG. 11B, lane 3). Examination of the profiles of total protein in Coomassie blue stained electrophoretograms of NIH 3T3 6-1 cell lysates did not reveal evident changes in the distribution of protein bands between the control and LO-treated cells (FIG. 1C) indicating that the enzyme did not oxidize proteins indiscriminately within the cell.

Inhibition by LO of FGF2-Stimulated Cell Cycle Progression

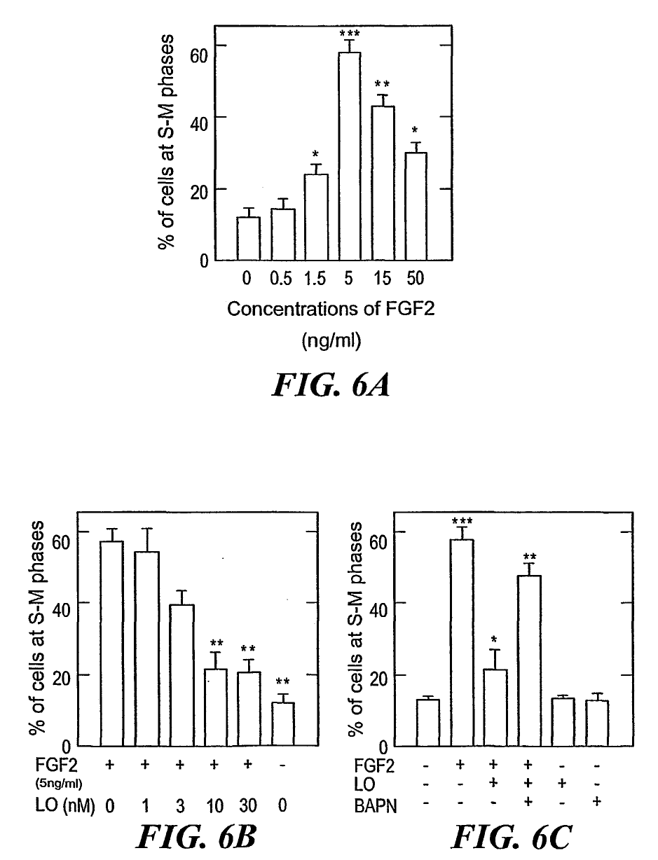

The activity of native and LO-oxidized FGF2 on cell cycle progression was evaluated by using fluorescence activated cell sorting (FACS) analysis (Darzynkiewicz et al.). Swiss 3T3 cells were growth-arrested by incubation in 0.3% fetal bovine serum (FBS)/Dulbecco's Modified Eagle's Medium (DMEM) for 3 days. As depicted in FIGS. 6A-6C, the prepared growth-arrested cells were treated for 20 h at 37° C. with or without FGF2, LO, BAPN or their combinations as indicated. After treatment, cells were washed, suspended by treatment with trypsin, washed again and fixed in 0.5% formaldehyde/0.1% Triton X-100/PBS for 15 minutes on ice followed by incubation of samples in 10 μg propidium iodide and 20 units RNAse per ml of PBS for 60 minutes in the dark for DNA staining. The fluorescence intensities of each sample of 10,000 cells in G₁, S and G₂/M phases were measured by flow cytometry at the FL2 setting of the fluorescence activated cell sorting (FACS) instrument (Becton-Dickinson Corp.). FIG. 6A shows a dose response of FGF2 in cell cycle progression. FIG. 6B shows LO inhibition of FGF2stimulated cell cycle progression (the control represents cells treated with 5 ng ml⁻¹ FGF2 in the absence of added LO). FIG. 6C shows effects of inactivation of LO by BAPN on FGF2-stimulated the cell cycle progression. LO was presented at 10 nM, FGF2 at 5 ng ml⁻¹ and BAPN at 100 μM. Data represent the means (+sd) of three separate experiments each assessed with duplicate dishes. Asterisks indicate values significantly different from the control as determined by ANOVA analysis: * p<0.05, ** p<0.01, *** p<0.001.

As shown in FIG. 6A, FGF2, alone, promoted the progression of growth-arrested Swiss 3T3 fibroblasts from the G0 and G1 to the S and G2/M phases. The maximum effect was seen at 5 ng FGF2/ml (0.28 nM FGF2) where the relative number of cells entering the S-M phases was 4.7-fold of that of the control. Cell cycle progression induced by bFGF was inhibited by increasing concentrations of LO (FIG. 6B). Stimulation obtained with 0.28 nM FGF2 maximally decreased by 80% at 10 nM (0.32 μg/ml) LO. β-Aminopropionitrile (BAPN; 100 μM), a specific, irreversible inhibitor of LO (Li et al., 1995; Kagan et al., 1995), effectively prevented LO from inhibiting FGF2-induced cell cycle progression (FIG. 6C). FGF2 treated with LO and BAPN induced 82±6% of the response observed with FGF2 alone. Thus, the inhibition by LO of the FGF2 stimulated cell cycle progression requires the catalytic function of LO. As noted, expression of the full biological effects of FGF2 requires its interaction with both HSPG receptors and tyrosine kinase receptors on the cell surface. The interactions of FGF2 with HSPG and its tyrosine kinase receptors are dependent upon a specific set of lysine residues on the surface of the growth factor including K26, K125 and K135 at HSPG binding sites and K110 at the FGF2 receptor binding domain (residues 106-115) (Eriksson et al.; Faham et al.). The inhibiting effect of LO on the induction of cell cycle progression may then reflect the oxidation of lysines required for the interaction of FGF2 with HSPG and receptors.

Suppression by LO of MAP Kinase Phosphorylation Stimulated by FGF2

Since activation of MAP kinases by FGF2 is one of the earliest mitogenic signaling events within cells (Maher), the effects of LO on FGF2-stimulated MAP kinase phosphorylation were tested by Western blot analysis using an antibody specific for phospho-extracellular signal-regulated kinases 1 and 2 (ERK1 and ERK2). The amount of total ERK1 and ERK2 MAP kinases within the same cell extracts was determined by using an antibody that recognizes both the phospho- and non-phospho-forms of ERK1 and ERK2. As shown in FIGS. 7A and B, growth-arrested Swiss 3T3 were untreated (lane 1) or treated with 5 ng ml⁻¹L FGF2 (lane 2), 5 ng ml⁻¹ FGF2+10 nM LO (lane 3), 5 ng ml⁻¹ FGF2+10 nM LO+100 μM BAPN (lane 4), 100 μM BAPN (lane 5), 10 nM LO (lane 6) or 200 μM genistein (lane 7) for 10 min. Cell extracts were prepared and equal amounts of proteins (25 μg) were analyzed by 10% SDS-PAGE. Immunoblotting was performed using antibodies specific for the active forms of ERK1 and ERK2 (A) and total ERKs (B).

As shown in FIG. 7A, tyrosine phosphorylation of both 42- and 44-kDa ERKs was markedly increased after stimulation of cells with 5 ng/ml FGF2 for 10 min (lane 2) in comparison to the control (lane 1). The levels of phospho-MAP kinases was reduced by concomitant treatment of cells with 5 ng/ml FGF2 and 10 nM LO (lane 3). The inhibitory effects of LO on FGF2 activation of MAP kinases was abolished by inhibition of LO catalysis by the inclusion of 100 μM BAPN. Whereas 100 μM BAPN, alone, did not affect the state of phosphorylation of MAP kinases in these growth arrested cells (lane 5), the presence of 10 nM LO in the absence of the addition of either BAPN or FGF2 slightly increased levels of phospho-ERK1/2 (compare lane 6 to the control in lane 1). MAP kinase activation by FGF2 was also inhibited by 200 μM genistein, a MAP kinase inhibitor (lane 7). None of these treatments exerted significant effects on the protein levels of total MAP kinases during the experimental period (FIG. 7B). These results are consistent with the suppressive effects of LO on the increase in cell cycle progression elicited by FGF2.

Inhibition of FGF2 Nuclear Localization by LO

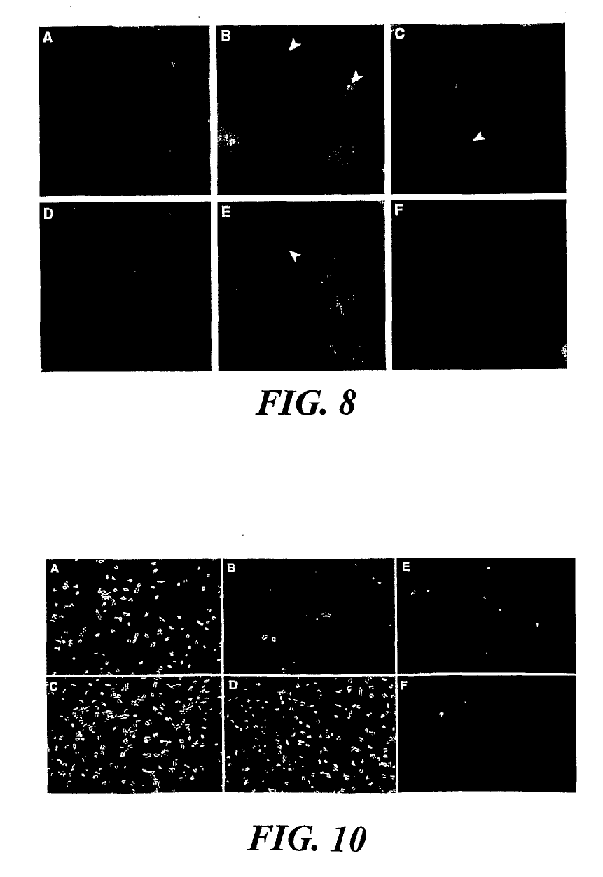

After endocytosis, a fraction of exogenous FGF2 can enter the nucleus of a variety of cell types in a cell cycle-dependent manner (Mason; Rifkin et al.; Prats et al.; Stachowiak et al., 1996; Arese et al.; Baldin et al.). Thus, the possibility that oxidation of FGF2 by LO affects the nuclear localization of this growth factor was tested by using immunochemistry. In FIGS. 8A-8F, growth arrested Swiss 3T3 cells on coverslips were treated without (A) or with 5 ng ml⁻¹ FGF2 (B), 5 ng ml⁻¹ FGF2+100 nM LO (C), 5 ng ml⁻¹ FGF2+10 nM LO+100 μM BAPN (D), 100 μM BAPN, alone (E), or 10 nM LO, alone (F) for 20 h at 37° C. The FGF2 in the cytoplasm and in the nuclei was revealed by immunochemical staining using monoclonal anti-FGF2 primary antibody followed by incubation with FITC-conjugated rabbit anti-mouse IgG. Omission of the primary antibody resulted in the absence of staining (not shown).

Growth-arrested cells exhibited a relatively minor degree of endogenous FGF2 immunoreactivity in the nucleus with greater amounts seen in the cytoplasm (FIG. 8A). Incubation of cells with 0.28 nM FGF2 for 1 h significantly increased nuclear staining evidenced by brightly stained nucleolar clusters (FIG. 8B) and consistent with results described previously (Arese et al.; Baldin et al.). In contrast, prominent FGF2 staining was observed only in the perinuclear region in cells, which had been simultaneously incubated with FGF2 and 10 nM LO. Moreover, these cells did not exhibit enhanced levels of intranuclear FGF2 (FIG. 8C). The inhibition of nuclear translocation of FGF2 by LO was prevented when 100 μM BAPN was included in the incubation medium with LO and FGF2. Under these circumstances, FGF2 was prominently accumulated within the nucleus (FIG. 8D). Inhibition of endogenous LO activity levels by incubation of cells with BAPN in the absence of added LO increased FGF2 transport to the nucleolus (FIG. 8E). Addition of LO in the absence of exogenous FGF2 or BAPN reduced levels of nuclear FGF2 (FIG. 8F). The oxidation of FGF2 by endogenous or exogenous LO inhibits the nuclear localization of FGF2 pointing to a mechanism for the LO-mediated inhibition of FGF2-stimulated cell cycle progression. Addition of exogenous 18 kDa FGF2 increased intranuclear FGF2 possibly as a result of the translocation of extracellular 18 kDa isoform (Baldin et al.) and/or the importing of intracellular larger isoforms (Arese et al.; Bikfalvi et al.). Inhibition of FGF2 nuclear uptake very likely reflects the LO-catalyzed generation of peptidyl AAS from key lysine residues in FGF2. As a consequence of LO-mediated oxidation, it is expected that FGF2 would no longer bind properly to its tyrosine kinase receptors and/or to HSPG sites and, thus, it would no longer induce the appropriate intracellular signal transduction responses. The accumulation of FGF2 in the perinuclear area when in the presence of LO suggests that the mechanisms of intracellular uptake of FGF2 were not compromised. Nevertheless, transport of intracellular bFGF into the nucleus was specifically disrupted (Arese et al.). It is possible that the H₂O₂ product of FGF2 oxidation by LO contributes to the suppression of the nuclear translocation of FGF2 since recent studies have shown that H₂O₂ can inhibit nuclear protein import (Czubbryt et al.).

In conclusion, the data presented indicate that FGF2, a key mitogen critical for normal and tumor cell growth, is a substrate of LO. The oxidation of FGF2 by LO resulted in the loss of much of its potential to activate ERK1/2 MAP kinases, to stimulate cell cycle progression and to be taken up into the nuclei of Swiss 3T3 cells. Moreover, incubation of LO with NIH 3T3 6-1 cells strongly inhibited the growth of these tumorigenic cells as a result of the LO-catalyzed oxidation of the FGF2-produced in excess by these cells. Thus these results illustrated a mechanism whereby LO functions as a suppressor of normal and excessive rates of cell proliferation by virtue of its modification of this growth factor.

Inhibition of the Growth of VEGF165-Stimulated VEC by LO

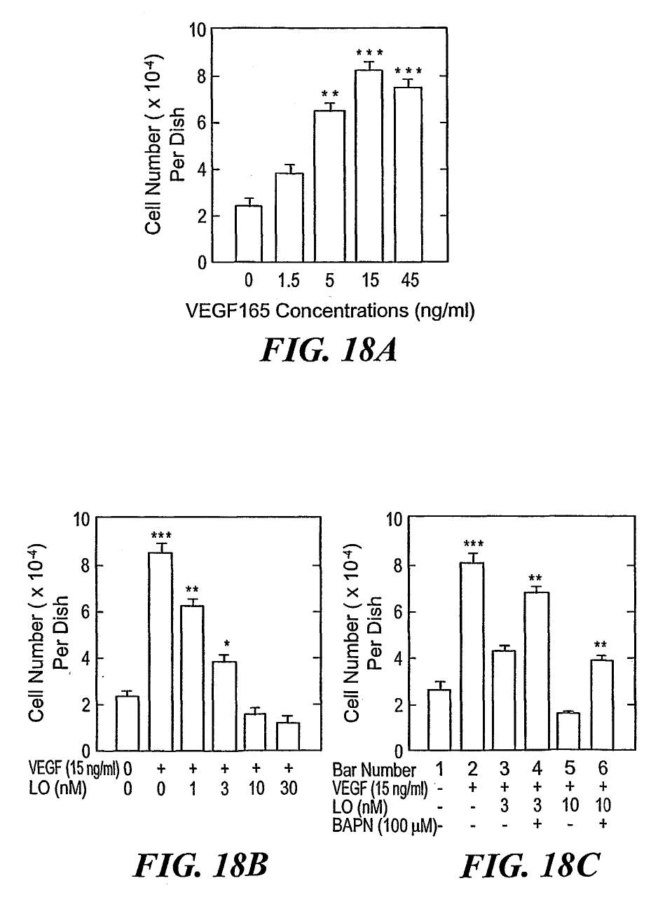

The effects of LO oxidized VEGF165 on cell proliferation was also assessed. VEGF165 is a potent mitogen for vascular endothelial cells (VEC). Porcine pulmonary arterial VEC (2×10⁴) were plated and incubated overnight in 35 mm-dishes containing 2 ml 15% FBS/M199 medium, and then refed with 5% FBS/M199 with different treatment regimens as shown in A, B and C of FIG. 18. FIG. 18A represents a dose response of VEGF165-stimulated cell growth. Media were supplemented with VEGF165 at the indicated doses and cells were incubated for additional 3 days. Cultures were refed once during the incubation period with fresh media supplemented with VEGF165 at indicated doses. Cells were harvested by trypsinization and counted with a hemacytometer. FIG. 18B represents the LO inhibition of VEGF165-stimulated cell growth. FIG. 18C represents the effects of inactivation of LO by BAPN on VEGF165-stimulated cell growth. LO: 3 or 10 nM, VEGF165: 15 ng ml⁻¹, BAPN: 100 μl. Data represent the means (±sd) of three separate experiments each assessed with triplicate dishes. In A and B, asterisks indicate values significantly different from the control as determined by ANOVA analysis: * p<0.05, ** p<0.01, *** p<0.001. In C, asterisks indicate values significantly different from the corresponding controls (i.e., bar 2 vs bar 1, bar 4 vs bar 3, and bar 6 vs bar 5) as determined by ANOVA analysis: ** p<0.01, *** p<0.001.

As shown in FIG. 18A, incubation of VEC with various doses of VEGF165 induced a dose-dependent stimulation of cell proliferation. Using the maximum stimulation of VEGF165 occurring at 15 ng/ml as control, the inclusion of LO (1-30 μM) in culture medium inhibited VEGF165-dependent cell growth with increased doses (FIG. 18B). LO inhibition of VEGF165-stimulated VEC proliferation was effectively blocked by 100 μM BAPN, an LO inhibitor (FIG. 18C). Thus, LO oxidation and inactivation of VEGF165 is the major mechanism for its inhibition of the growth of VEC incubated with this growth factor.

Inhibition by LO of PDGF-AB-Stimulated Cell cycle Progression

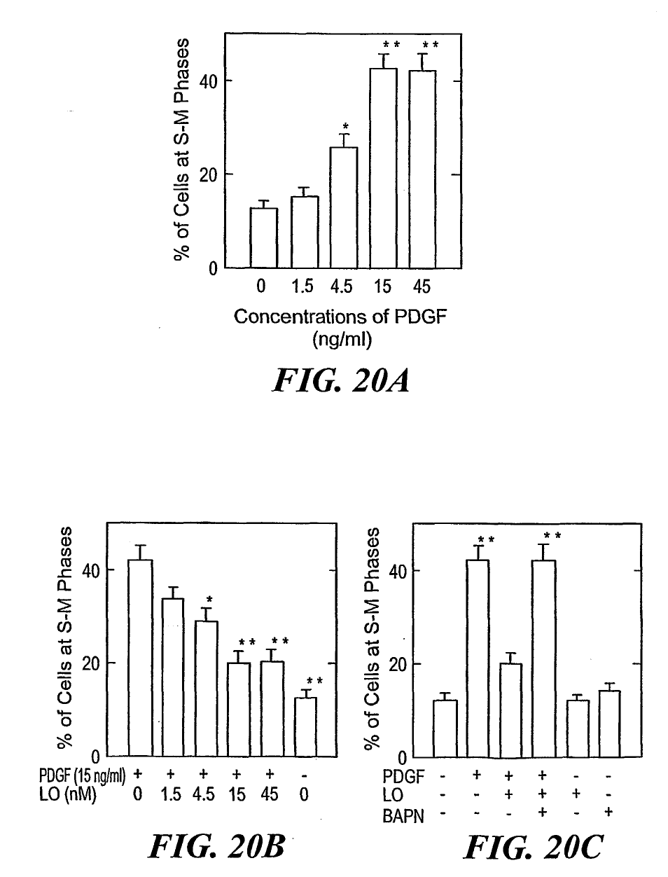

The effects of native and LO-oxidized PDGF-AB on Swiss 3T3 cell cycle progression revealed by fluorescence activated cell sorting (FACS) analysis (Darzynkiewicz et al.) was tested. Growth-arrested Swiss 3T3 cells were incubated for 20 h at 37° C. with or without PDGF-AB, LO, BAPN or their combinations as indicated. After incubation, FACS analysis was carried out to characterize the cell cycle progression under different conditions as described in FIG. 6. FIG. 20A represents a dose response of PDGF-AB in cell cycle progression. FIG. 20B represents the LO inhibition of PDGF-AB-stimulated cell cycle progression (the control represents cells treated with 15 ng ml⁻¹ PDGF-AB in the absence of added LO). FIG. 20C represents the effects of inactivation of LO by BAPN on PDGF-AB-stimulated the cell cycle progression. LO: 15 nM, PDGF-AB: 15 ng ml⁻¹, BAPN: 100 AM. Data represent the means (±sd) of three separate experiments each assessed with triplicate dishes. Asterisks indicate values significantly different from the control as determined by ANOVA analysis: * p<0.05, ** p<0.01, *** p<0.001.

FIG. 20A show that the PDGF-AB alone promoted the progression of growth-arrested Swiss 3T3 fibroblasts from the G0 and G1 to the S and G2/M phases. The maximum effect was seen at 15 ng PDGF-AB/ml (0.56 nM) where the effect on the cells entering the S-M phases was 3.3-fold of that of the control. Cell cycle progression induced by 0.56 nM PDGF-AB was markedly inhibited by increasing concentrations of LO (FIG. 20B) as evidenced by a 73% decrease at 15 nM (0.48 μg/ml) LO. As shown in FIG. 20C, 100 μM BAPN inactivated the catalytic activity of LO, thus effectively protected the stimulating effects of 0.56 nM PDGF on the cell cycle progression reaching 100.4% of the level stimulated with PDGF-AB alone. These results indicated that oxidation of PDGF-AB by LO inhibited its mitogenic effects on cell proliferation.

Inhibition by LO of Tat-Stimulated Vascular Endothelial Cells Growth

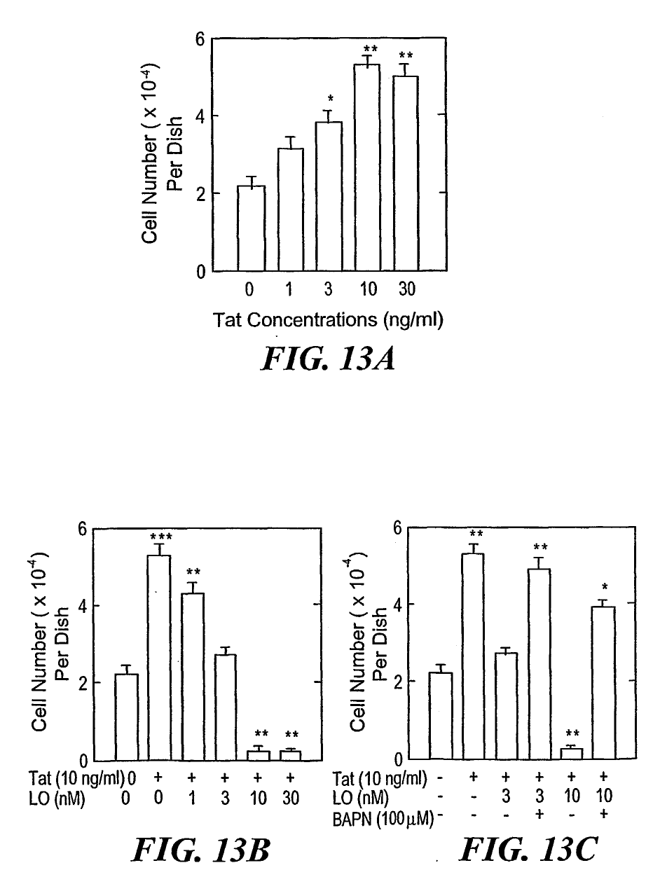

Stimulation of cell proliferation of porcine pulmonary arterial vascular endothelial cells (VEC) was tested with LO-oxidized Tat. VEC (2×10⁴) were plated and incubated overnight in 35 mm-dishes containing 2 ml 15% FBS/M199 medium, and then refed with 5% FBS/M199 with different treatment regimens as shown in A, B and C of FIG. 13. FIG. 13A represents a dose response of Tat-stimulated cell growth. Media were supplemented with Tat at the indicated doses and cells were incubated for additional 3 days. Cultures were refed once during the incubation period with fresh media supplemented with Tat at indicated doses. Cells were harvested by trypsinization and counted with a hemacytometer. FIG. 13B represents the LO inhibition of Tat-stimulated cell growth. FIG. 13C represents the effects of inactivation of LO by BAPN on Tat-stimulated cell growth. LO: 3 or 10 nM, Tat: 10 ng ml⁻¹, BAPN: 100 μM. Data represent the means (±sd) of three separate experiments each assessed with triplicate dishes. Asterisks indicate values significantly different from the control as determined by ANOVA analysis: * p<0.05, ** p<0.01, p<0.001.

As shown in FIG. 13A, exposure of VEC to Tat induced a dose-dependent increase in the number of cells incubated for 3 days in 5% FBS/M199 medium. The maximum stimulation of cell proliferation by Tat occurred at 10 ng/ml. Thus, this optimum dose of Tat was used as control to test LO inhibitory effects on Tat. As shown in FIG. 13B, Tat preincubated with various doses of LO lost its ability to promote cell growth in a dose-dependent manner. LO at 3 nM effectively suppressed the Tat-dependent cell proliferation to the control level. LO at doses ≧10 nM resulted in severe loss in cell numbers below the control. As shown in FIG. 13C, this effect of LO was totally inhibited by BAPN.

Numerical and morphological alterations in Tat-stimulated VEC cells treated with LO were observed under phase-contrast microscopy (25'). Cells were cultured as described in FIG. 13 and treated with additives as indicated below. The following represents FIG. 14 phase contrast microscopy: A, control; B, 10 ng ml⁻¹Tat; C, 10 ng ml⁻¹ Tat+3 nM LO; D, 10 ng ml⁻¹ Tat+3 nM LO that was preincubated with 100 μM BAPN for 15 min at 370; E, 100 μM BAPN, alone, and F, 3 nM LO, alone.

As exemplified in FIG. 14, phase-contrast microscopy confirmed inhibition of cell proliferation of Tat stimulated VEC by LO. As shown in FIG. 14B, after incubation for 3 days in the presence of 10 ng/ml Tat, the cells grew to confluence. In contrast, FIG. 14A showed control cells incubated under the same conditions grew to inconfluence. Preincubated with 3 nM LO for 30 min at 37° C., the growth stimulating ability of Tat was inhibited as shown in FIG. 14C. As shown in FIG. 14D, cells sustained growth to confluence in Tat preincubated with BAPN (100 μM) inactivated LO. Incubation with BAPN alone had no effect on the cell growth, as shown in FIG. 14E. FIG. 14F displayed cell morphological alterations in the presence of LO alone. These results indicated that LO anti-proliferative effects on Tat-stimulated VEC growth required the expression of its catalytic activity. Furthermore, the loss of biological functions of Tat upon LO action indicates that this enzyme may be an effective transactivation inhibitor to HIV-1.

Inhibition of the Angiogenic Response of VEC Stimulated by Tat on Matrigel by LO

VEC grown on Matrigel, a solid gel of basement membrane proteins, rapidly align and form capillary-like structures providing a convenient in vitro model for angiogenesis studies (Albini et al., 1995; Grant et al.). Tat has been reported to enhance this in vitro morphogenesis of VEC consistent with its angiogenic property (Albini et al., 1995). Since Tat has now been shown to be a substrate of LO, the inhibitory effects by LO of angiogenic response of Tat-stimulated VEC on Matrigel were further studied.

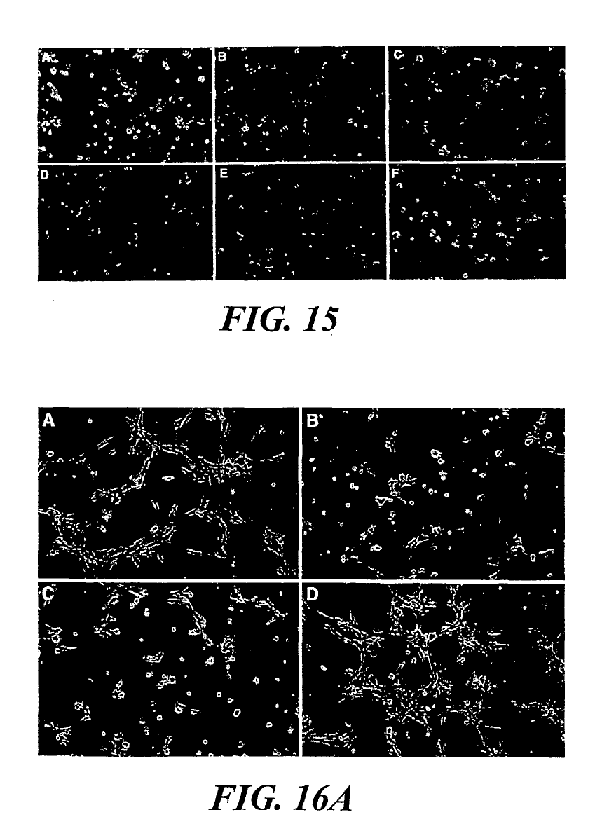

Cells (3×10⁵) were plated on Matrigel-coated dishes. After 1 h incubation, various reagents were added to each culture. Cells were incubated for additional 3 h and the angiogenic response of Tat-stimulated VEC on Matrigel was revealed by phase-contrast microscopy (25×). The following represents FIG. 15 phase-contrast microscopy: A, control; B, 10 ng ml⁻¹ Tat; C, 10 ng ml⁻¹ Tat+10 nM LO; D, 10 ng ml⁻¹ Tat+10 nM LO that was preincubated with 100 μM BAPN for 15 min at 370; E, 100 μM BAPN, alone, and F, 10 nM LO, alone.

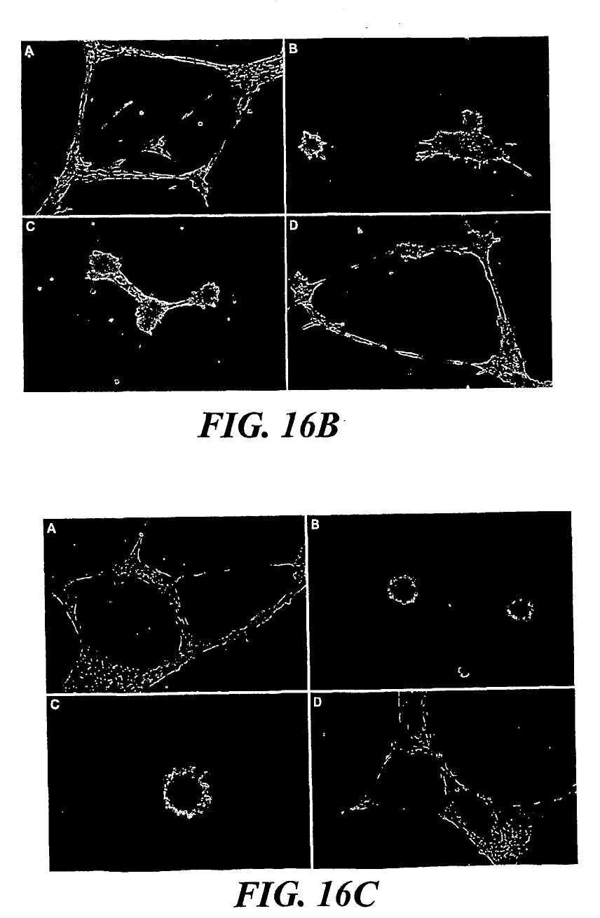

VEC incubation on Matrigel in the presence of Tat (1 h preincubation and 3 h Tat exposure) facilitated the formation of capillary like structures as shown in FIG. 15B (compared to the control in FIG. 15A). At least 7 h-incubation of VEC without Tat was required to exhibit the appearance of the same angiogenic response (compare FIG. 15B with FIG. 16Aa). As shown in FIG. 15C, LO effectively blocked this Tat-stimulated morphogenesis of VEC. However, this LO-mediated inhibition of the angiogenic response in vitro was not prevented by BAPN, as shown in FIG. 15D. FIG. 15E and FIG. 15F exhibited VEC on Matrigel treated with BAPN or LO, alone. Thus, LO inhibition of the growth and the differentiation of VEC may be mediated by different LO-dependent mechanisms.

Time course studies on inhibition by LO of angiogenic response of VEC on Matrigel without Tat stimulation was also investigated. Cells (3×10⁵) were plated on Matrigel-coated dishes. After 1 h incubation, various reagents were added to each culture. As shown in FIG. 16, cells were incubated for additional 6 (A), 24 (B) and 48 (C) h and the angiogenic response of VEC on Matrigel was revealed by phase-contrast microscopy (25×). At each time point, a, control; b,10 nM LO; c,10 nM LO that was preincubated with 100 μM BAPN for 15 min at 37°; d, 100 μM BAPN, alone.

As illustrated, active (FIGS. 16Ab, 16Bb and 16Cb) and BAPN-inactivated (FIGS. 16Ac, 16Bc and 16Cc) LO both exhibited strong inhibitory effects while BAPN, alone, (FIGS. 16Ad, 16Bd and 16Cd) did not have significant effects on the organization of VEC to form capillary-like structure on Matrigel under these conditions. These findings suggest that the intrinsic sequence and/or protein structure of the enzyme may account for this anti-angiogenic effect of LO. An analysis of the LO sequence indicated that LO contains a binding domain for cell adhesion molecules located on cell surfaces (Ruker et al.; Saux et al.). Thus, LO may directly target endothelial cells by virtue of binding to these cell surface molecules effectively modulating cell behavior.

In conclusion, the data presented indicate that the unique catalytic activity of LO targets not only polypeptides synthesized by mammalian cells but also those derived from microorganisms. HIV-1 Tat, a virus encoded protein as the first case, was demonstrated as an effective substrate of LO. Although LO inhibition of Tat-stimulated cell proliferation was dependent on its catalytic activity while LO interference with Tat-enhanced angiogenic response of VEC on Matrigel was independent on its catalytic activity, both properties of LO may coordinately contribute to the anti-angiogenic/tumorigenic potential of this enzyme. This is the critical basis for development of LO and related products as therapeutics for AIDS-KS, an angioproliferative disease.

In light of the newly discovered unique catalytic properties of LO toward multiple factors, which share common structural features and are intrinsically involved in the mechanisms of AIDS and AIDS-KS pathogenesis, LO, the matrix crosslinking enzyme, can be developed as an effective suppressor of AIDS and AIDS-KS by virtue of its oxidation and thereby inactivation of such multiple “substrates” as Tat, FGF2 and VEGF, the key pathogenic factors for AIDS-KS onset. In HIV-1 infected cells, LO inactivation of Tat, a critical transactivator for viral gene expression, should directly inhibit HIV-1 replication. In HIV-1 negative, uninfected cells such as endothelial or KS cells, Tat inactivation by LO should block its interaction with target cells and reduce the production of inflammatory cytokines, growth factors and angiogenic molecules, thus abolishing its potential for growth stimulation and angiogenesis. Moreover, LO inactivation of FGF2 and VEGF should also disrupt auto-and/or paracrine loops, a vital mechanism for sustaining tumor growth, resulting in regression of AIDS-KS including even those at the late nodular stages.

### Use

Prevention and Treatment of Conditions with Lysyl Oxidase Treatments

Prevention and treatment of exemplary diseases manifested by aberrant cell growth control for use in accordance with the present invention may include, inter alia, Tumors such as breast cancer, colon cancer, renal cancer, prostate cancer, ovarian cancer, uterine cancer, lung cancer, brain cancer, skin cancer, embryo carcinoma, teratocarcinoma, germ cell tumor, osteocarcoma, fibrosacoma, malanoma, hemangioma, angiofibroma, angioendothelioma and AIDS-associated malignancies including Kaposi's sarcoma.

Exemplary diseases manifested by aberrant growth control, other than tumors, may include those angiogenic diseases such as neovascular glaucoma, angiomatosis, and hyperplastic diseases such as thyroid hyperplasia associated with elevated levels of FGF2 and FGF receptors in idiopathic multinodular goiter (see, for example, Thompson et al.), and giant cell arteritis (GCA) accompanied by overexpressing PDGF A and PDGF B in polymyalgia rheumatica (PMR) (see, for example, Kaiser et al.), etc.

Exemplary diseases manifested by neither tumors nor angiogenic and hyperplastic diseases, may include AIDS and people with HIV-1 infection without AIDS.

These and other diseases may be prevented and/or treated by the growth inhibitor (or transactivation inhibitor to HIV-1) of the present invention, which may be used to modify growth/angiogenic factors to reduce the mitogenic and angiogenic potential to both normal and tumor cells and also to modify HIV-1 Tat to reduce the transcriptional potential to HIV-1.

The therapeutic compositions of the invention can be administered locally, orally, topically, or parenterally (e.g., intranasally, subcutaneously, intramuscularly, intravenously, or intra-arterially) by routine methods in pharmaceutically acceptable inert carrier substances. For example, the compositions of the invention may be administered in a sustained release formulation using a biodegradable biocompatible polymer, or by on-site delivery using micelles, gels or liposomes. Treatments may also be genetically engineered with a carrier protein or antibody, cytokines, specific cell surface receptors, or cell adhesion molecules.

For example, liposomally encapsulated LO and related LO products can specifically target the tumor cells or HIV-1 infected cells such as CD4+ T lymphocytes. This can be accomplished by inserting ligands on the membrane of liposomes that bind only to receptors known to be expressed on the surface of the target cells, employing standard techniques in genetic engineering. The pharmacological effects of LO can also be derived as a result of appropriate gene therapy. Vectors containing the LO gene can be introduced into the target cells or tissues where the LO gene products is to be produced in situ thus interfering with internal autocrine growth factors or HIV-1 Tat by oxidation and inactivation of these proteins. Moreover, since CD34+ hematopoietic stem cells can differentiate into the various cell types such as T cells and monocytes involved in HIV-1 pathogenesis, these CD34+ cells can be used as target cells for transfection of the LO gene in vitro. Once produced, these LO expressing stem cells can be infused into AIDS patients or HIV-1 infected persons where they would differentiate into T cells or monocytes which would then be resistant to HIV-1 due to their endogenous production of LO, an anti-transactivation inhibitor of HIV-1. This approach should prove to be of great benefit for the treatment and prevention of HIV-1 infection and its consequences.

The cell growth factor inhibitor can be administered in a dosage of 0.1 μg/kg/day to 10 μg/kg/day, preferably 0.5 μg/kg/day to 5 μg/kg/day, and preferably 1 μg to 100 μg for local administration. Optimal dosage and modes of administration can readily be determined by conventional protocols.

The therapeutic compositions of the invention can be comprised in a kit with instructions for use thereof.

The therapeutic compositions of the invention can be administered independently or co-administered with another antineoplastic agent. The therapeutic compositions of the invention will be particularly useful when co-administered with several cytokines such as TGF-β1 and IL-1β and hormones such as testosterone and estrogen, which elevate the production of endogenous LO, as well as trace copper (Cu²⁺), a cofactor of LO, thus facilitating the pharmacological effects of exogenous LO. LO and LO fragments can be used as conjugates, and therefore, carriers of anti-tumor drugs or toxins to more effectively target and kill tumor cells. Properties of the binding domain for cytokines, growth hormones and adhesion molecules on the cell surface in the LO sequence allow for such conjugation of drugs or toxins.

### REFERENCES

Albini, A., Barillari, G., Benelli, R., Gallo, R. C. and Ensoli, B. (1995). Angiogenic properties of human immunodeficiency virus type 1 Tat protein. Proc. Natl. Acad. Sci. 92,4838-4842.

Albini, A., Benelli, R., Presta, M., Rusnati, M., Ziche, M., Rubartelli, A., Paglialunga, G., Bussolino, F. and Noonan, D. (1996). HIV-tat protein is a heparin-binding angiogenic growth factor. Oncogene 12,289-297.

Albini, A., Fontanini, G., Masiello, L., Tacchetti, C., Bigini, D., Luzzi, P., Noonan, D. M. and Stetler-Stevenson, W. G. (1994). Angiogenic potential in vivo by Kaposi's sarcoma cell-free supernatants and HIV-1 tat product: inhibition of KS-like lesions by tissue inhibitor of metalloproteinase-2. AIDS 8,1237-1244.

Ambs, S., Bennett, W. P., Merriam, W. G., Ogunfusika, M. O., Oser, S. M., Khan, M. A., Jones, R. T. and Harris, C. C. (1998). Vascular endothelial growth factor and nitric oxide synthase expression in human lung cancer and the relation to p53. Br. J. Cancer 78,233-239.

Antman, K. and Chang Y. (2000). Kaposi's sarcoma. New Eng. J. Med. 342,1027-1038.

Arese, M., Chen, Y., Florkiewicz, R. Z., Gualandris, A., Shen, B. and Rifkin, D. B. (1999). Nuclear activities of basic fibroblast growth factor: potentiation of low-serum growth mediated by natural or chimeric nuclear localization signals. Mol. Biol. Cell 10, 1429-1444.

Arora, N., Masood, R., Zheng, T., Cai, J., Smith, D. L. and Gill, P. S. (1999). Vascular endothelial growth factor chimeric toxin is highly active against endothelial cells. Cancer Res. 59,183-188.

Baldin, V., Roman, A. M., Bosc-Bierne, I., Amalric, F. and Bouche, G. (1990). Translocation of bFGF to the nucleus is G1 phase cell cycle specific in bovine aortic endothelial cells. EMBO J. 9,1511-1517.

Barillari, G., Gendelman, R., Gallo, R. C. and Ensoli, B. (1993). The Tat protein of human immunodeficiency virus type 1, a growth factor for AIDS Kaposi's sarcoma and cytokine-activated vascular cells, induces adhesion of the same cell types by using integrin receptors recognizing the RGD amino acid sequence. Proc. Natl. Acad. Sci. USA 90,7941-7945.

Barillari, G., Sgadari, C., Palladino, C., Gendelman, R., Caputo, A., Morris, C. B., Nair, B. C., Markham, P., Nel, A., Sturzl, M. and Ensoli, B. (1999). Inflammatory cytokines synergize with the HIV-1 Tat protein to promote angiogenesis and Kaposi's sarcoma via induction of basic fibroblast growth factor and the αvβ3 integrin. J. Immunol. 163,1929-1935.

Basilico, C. and Moscatelli, D. (1992). The FGF family of growth factors and oncogeges. Adv. Cancer Res. 59,115-165.

Becker, D., Meier, C. B. and herlyn, M. (1989). Proliferation of human malignant melanomas is inhibited by antisense oligodeoxynucleotides targeted against human fibroblast growth factor. EMBO J. 8,3685-3691.

Besser, D., Presta, M. and Nagamine, Y. (1995). Elucidation of a signaling pathway induced by FGF2 leading to uPA gene expression in NIH 3T3 fibroblasts. Cell Growth Differ. 6,1009-1017.

Betsholtz, C., Johnsson, A., Heldin, C-H., Westermark, B., Lind, P., Urdea, M. S., Eddy, R., Shows, T. B., Philportt, K., Mellor, A. L., Knott, T. J. and Scott, J. (1986). cDNA sequence and chromosomal localization of human platelet-derived growth factor A chain and its expression in tumor cell lines. Nature 320, 695-699.

Bieniasz, P. D., Grdina, T. A., Bogerd, H. P. and Cullen, B. R. (1999). Analysis of the effect of natural sequence variation in Tat and in cyclin T on the formation and RNA binding properties of Tat-cyclin T complex. J. Virol. 0.73,5777-5786.

Bikfalvi, A., Klein, S., Pintucci, G., Quarto, N., Mignatti, P. and Rifkin, D. B. (1995). Differential modulation of cells phenotype by different molecular weight forms of basic fibroblast growth factor: possible intracelluar signaling by the high molecular weight forms. J. Cell Biol. 129,233-243.

Bohan-Morris, C., Gendelman, R., Marrogi, A. J., Lu, M., Lockyer, J. M., Alpenrein-Lea, W. (1996). Immunohistochemical detection of bcl-2 in AIDS-associated and classical Kaposi's sarcoma. Am. J. Pathol. 148,1055-1063.

Borgstrom, P., Bourdon, M. A., Hillan, K. J., Sriramarao, P. and Ferrara, N. (1998). Neutralizing anti-vascular endothelial growth factor antibody completely inhibits angiogenesis and growth of human prostate carcinoma microtumors in vivo. Prostate 35,1-10.

Bovi, P. D., Donti, E., Knowles II, D. M., Friedman-Kien, A., Luciw, P. A., Dina, D., Dalla-Favera, R. and Basilico, C. (1986). Presence of chromosomal abnormalities and lack of AIDS retrovirus DNA sequences in Kaposi's sarcoma. Cancer Res. 46,6333-6338

Bower, M., Fox, P., Fife, K., Gill, J., Nelson, M., and Gazzard, B. (1999). Highly active anti-retroviral therapy (HAART) prolongs time to treatment failure in Kaposi's sarcoma. AIDS 13,2105-2111.

Browder, T. M, Dunbar, C. E. and Nienhuis, A. W. (1989). Private and public autocrine loops in neoplastic cells. Cancer cell 1,9-17.

Browder, T. M., Abrams, J. S., Wong, P. M. and Nienhuis, A. W. (1989). mechanism of autocrine stimulation in hematopoietic cells producing interleukin-3 after retrovirus-mediated gene transfer. Mol. Cell. Biol. 9,204-213.

Brown, L. F., Tognazzi, K., Dvorak, H. F. and Harrist, T. J. (1996). Strong expression of kinase insert domain-containing receptor, a vascular permeability factor/vascular endothelial growth factor receptor in AIDS-associated Kaposi's sarcoma and cutaneous angiosarcoma. Am. J. Pathol. 148,1065-1074.

Buonaguro, L., Barillari, G., Chang, H. K., Bohan, C. A., Kao, V., Morgan, R., Gallo, R. C. and Ensoli, B. (1992). Effects of the human immunodeficiency virus type 1 Tat protein on the expression of inflammatory cytokines. J. Virol. 66,7159-7167, 1992.

Burgess, W. H. and Maciag, T. (1989). The heparin-binding (fibroblast) growth factor family of proteins. Annu. Rev. Biochem. 58,575-606.

Calnan, B. J., Biancalana, S., Hudson, D. and Frankel, A. D. (1991). Analysis of arginine-rich peptides from the HIV Tat protein reveals unusual features of RNA-protein recognition. Genes Dev. 5,201-210.

Carroll, R., Martarano, L. and Derse, D. (1991). Identification of lentivirus tat functional domains through generation of equine infections anemia virus/human immunodeficiency virus type 1 tat gene chimeras. J. Virol. 65,3460-3467.

Chandler, L., Sosnowski, B. A., Greenlees, L., Aukerman, S. L., Baird, A. and Pierce, G. F. (1999). Prevalent expression of fibroblast growth factor (FGF) receptors and FGF2 in human tumor cell lines. Int. J. Cancer 81,451-458.

Chang, H-K, Gallo, R. C., and Ensoli, B. (1995). Regulation of cellular gene expression and function by human immunodeficiency virus type 1 Tat protein. J. Biomed. Sci. 2,189-202.

Chang, Y., Cesarman, E., Pession, M. S., Lee, F., Culpepper, J., Knowles, D. M., and Moore, P. S. (1994). Identification of herpesvirus-like DNA sequense in AIDS-associated Kaposi's sarcoma. Science 266,1865-1869.

Contente, S., Kenyou, K., Rimoldi, D. and Friedman, R. M. (1990). Expression of gene rrg is associated with reversion of NIH 3T3 transformed with LTR-c-Ha-ras. Science 249,796-798.

Czubayco, F., Liaudet-Coopman, E. D. E., Aigner, A., Tuveson, A. T., Berchem, G. J. and Wellstein, A. (1997). A secreted FGF-binding protein can serve as the angiogenic switch in human cancer. Nature Med. 3,1137-1140.

Czubbryt, M. P., Austria, J. A. and Pierce, G. N. (2000). Hydrogen peroxide inhibition of nuclear protein import is mediated by the mitogen-activated protein kinase, ERK2. J. Cell Biol. 148, 7-15.

Darzynkiewicz, Z. (1994). Cell cycle analysis by flow cytometry. In Celis, J. E. (ed). Cell Biology, A Laboratory handbook. Vol. 1, pp261-271, Academic Press, San Diego. Derse, D., Carvakho, M., Carroll, R. and Peterlin, B. M. (1991). A minimal lentivirus tat. J. Virol. 65,7012-7015.

Devries, C., Escobedo, J. A., Ueno, H., Houck, K., Ferrara, N. and Williams, L. T. (1992). The fms-like tyrosine kinase, a receptor for vascular endothelial growth factor. Science 255,989-991.

Dezube, B. J., von Roenn, J. H., Holden-Wiltse, J., Cheung, T. W., Remick, S. C., Cooley, T. P., Moore, J., Sommadossi, J-P., Shriver, S. L., Suckow, C. W. and Gill, P. S.(1998). A phase I dose escalation and pharmacokinetics study of TNP-470- in AIDS-related Kaposi's sarcoma (ACTG-215). J. Clin. Oncol. 16,1444-1449.

Donato, A. D., Lacal, J. L., Duca, M. D., Giampuzzi, M., Ghiggeri, G. and Gusmano, R. (1997). Micro-injection of recombinant lysyl oxidase blockes oncogenic p21-Ha-Ras and progesterone effects on Xenopus Laevis oocyte maturation. FEBS Lett. 419,63-68.

Endo, S., Kubota, S., Siomi, H., Adachi, A., Oroszlan, S., Maki, M., and Hatanaka, M. (1989). A region of basic amino acid cluster in HIV-1 Tat protein is essential for trans-acting activity and nuclear localization. Virus Genes 3,99-110.

Ensoli, B. and Sturzl, M. (1998). Kaposi's Sarcoma: A result of interplay among inflammatory cytokines, angiogenic factors and viral agents. Cytokine & Growth Factor Rev. 9,63-83.

Ensoli, B., Buonaguro, L., Barillari, G., Fiorelli, V., Gendelman, R., Morgan, R. A., Wingfield, P. and Gallo, R. C. (1993). Release, uptake, and effects of extracellular human immunodeficiency virus type 1 Tat protein on cell growth and viral transactivation. J. Virol. 67,277-287.

Ensoli, B., Gendelman, R., Markham, P., Fiorelli, V., Colombini, S., Raffeld, M., Cafaro, A., Chang, H. K., Brady, J. N. and Gallo, R. C. (1994). Synergy between basic fibroblast growth factor and HIV-1 Tat protein in induction of Kaposi's sarcoma. Nature 371,674-680.

Ensoli, B., Markham, P., Kao, V., Barillari, G., Fiorelli, V., Gendelman, R., Raffeld, M., Zon, G. and Gallo, R. C. (1994). Block of AIDS-Kaposi's sarcoma (KS) cell growth, angiogenesis and lesion formation in nude mice by antisense oligonucleotides targeting basic fibroblast growth factor: a novel strategy for the therapy of KS. J. Clin. Invest. 94,1736-1746.

Ensoli, B., Nakamura, S., Salahuddin, S. Z., Biberfeld, P., Larsson, L., Beaver, B., Wong-Staal, F. and Gallo, R. C. (1989). AIDS-Kaposi's sarcoma-derived cells express cytokines with autocrine and paracrine growth factor effects. Science 2443,223-226.

Eriksson, A. E., Cousens, L. S., Weaver, L. H. and Matthews, B. W. (1991). Three-dimensional structure of human basic fibroblast growth factor. Proc. Natl. Acad. Sci. USA 88,3441-3445.

Eva, A., Robbins, K. C., Andersen, P. R., Srinivasan, A., Tronick, S. R., Reddy, E. P., Ellmore, N. W., Galen, A. T., Lautenberger, J. A., Papas, T. S., Westin, E. H., Wong-Staal, F., Gallo, R. C. and Aaronson, S. A. (1982). Cellular genes analogous to retroviral onc genes are transcribed in human tumor cells. Nature (Lond.) 295,116-119.

Faham, S., Hileman, R. E., Fromm, J. R. Linhardt, R. J. and Rees, D. C. (1996). Heparin structure and interactions with basic fibroblast growth factor. Science 271, 1116-1120.

Florkiewicz, R. Z. and Sommer, A. (1989). Human basic fibroblast growth factor gene encodes four polypeptides: three initiate translation from non-AUG codons. Pro. Natl. Acad. Sci. USA 86,3978-3981.

Folkman, J. and Klagsbrun, M. (1987). Angiogenic factors. Science 235,442-447.

Frankel, A. D., Chen, L., Cotter, R. J. and Pabo, C. O. (1988) Dimerization of the tat protein from human immunodeficiency virus: a cysteine-rich peptide mimics the normal metal-linked dimer interface. Proc. Natl. Acad. Sci. USA 85,6297-6300.

Fujimoto, J., Sacaguchi, H., Hirose, R., Ichigo, S. and Tamaya. T. (1999). Expression of vascular endothelial growth factor (VEGF) and its mRNA in uterine cervical cancers. Br. J. Cancer 80,827-833.

Ganju, R. K., Munshi, N., Nair, B. C., Liu, Z-Y., Gill, P., Groopman, J. E. (1998). Human immunodeficiency virus Tat modulates the Flk-1/KDR receptor, mitogen activated protein kinases, and components of focal adhesion in Kaposi's sarcoma cells. J. Virol. 72,6131-6137.

Garcia, J. A., Harrich, D., Pearson, L., Mitsuyasu, R. and Gaynor, R. B. (˜1988). Functional domains required for tat-induced transcriptional activation of the HIV-1 long terminal repeat. EMBO J. 7,3143-3147.

Gill, P. S., Akil, B., Colletti, P., Parick, M., Loureiro, C., Bernstein-Singer, M., Krailo, M. and Levine, A. M., (1989). Pulmonary Kaposi's Sarcoma. Clinical findings and results of therapy. Am. J. Med. 87,57-61

Gnann, Jr., J. W., Pellett, P. and Jaffe, H. W. (2000). Human herpesvirus 8 and Kaposi's sarcoma in persons infected with human immunodeficiency virus. Clin. Infec. Dis. 30,S72-S76.

Grant, D. S., Tashiro, K-I., Segui-Real, B., Yamada, Y., Martin, G. R. and Kleinman, H. K. (1989). Two different laminin domains mediate the differentiation of human endothelial cells into capillary-like structures in vitro. Cell 58,933-943.

Grotendorst, G. R., Chang, T., Seppa, H. E., Kleinman, H. K. and Martin, G. R. (1982). Platelet-derived growth factor is a chemoattractant for vascular smooth muscle cells. J. Cell. Physiol. 113, 261-266.

Halaban, R., Langdon, R., Birchall, N., Cuono, C., Baird, A., Scott, G., Moellmann, G. and McGuire, J. (1988). Basic fibroblast growth factor from human keratinocytes is a natural mitogen for melanocytes. J. Cell Biol. 107,1611-1619.

Hammacher, A., Hellman, U., Johnsson, A., Ostman, A., Gunnarsson, K., Westermark, B., Wasteson, A. and Heldin, C-H. (1988). A major part of platelet-derived growth factor purified from human platelets is a heterodimer of one A and one B. J. Biol Chem. 263,16493-16498.

Harrington, Jr., W., Sieczkowski, L., Sosa, C., Chan-a-Sue, S., Cai, J. P., Cabral, L. and Wood, C. (1997). Activation of HHV-8 by HIV-1 Tat. Lancet 349,774-775.

Harris, E. D. (1993). Menkes' disease: perspective and update on a fatal copper disorder. Nutr. Rew. 51,235-245.

Heldin, C-H. (1992). Structural and functional studies on platelet-derived growth factor. EMBO. J. 11, 4251-4259.

Heldin, C-H. (1995). Dimerization of cell surface receptors in signal transduction. Cell 80,213-223.

Heldin, C-H., Waasteson, A. and Westermark, B. (1985). Platelet-derived growth factor. Mol. Cell. Endo. 39,169-187.

Heldin, C-H., Westermark, B. and Wasteson, A. (1980). Chemical and biological properties of a growth factor from human-cultured osteosarcoma cells: resemblance with platelet-derived growth factor. J. Cell. Physiol. 105,235-246.

Herbert, J-M., Bono, F. and Savi, P. (1996). The mitogenic effects of H₂O₂ for vascular smooth muscle cells is mediated by an increase of the affinity of basic fibroblast growth factor for its receptor. FEBS Lett. 395,43-47.

Hermansson, M., Funa, K., Hartman, M., Claesson-Welsh, L., Heldin, C-H., Westermark, B. and Nister, M. (1992). Platelet-derived growth factor and its receptors in human glioma tissue: expression of messenger RNA and protein suggests the presence of autocrine and paracrine loops. Cancer Res. 52,3213-3219.

Jeang, K-T., Xiao, H. and Rich, E. A. (1999). Multifaceted activities of the HIV-1 transactivator of transcription, Tat. J. Biol. Chem. 274,28837-28840.

Johnsson, A., Heldin, C-H., Wasteson, A., Westermark, B., Deuel, T. F., Huang, J. S., Seeburg, P. H., Gray, A., Ullrich, A., Scrace, G., Stroobant, P and Waterfield, D. (1984). The c-sis gene encodes a precursor of the B chain of platelet-derived growth factor. EMBO. J. 3, 921-928.

Johnsson, A., Heldin, C-H., Westermark, B. and Wasteson, A. (1982). Platelet-derived growth factor: identification of constituent polypeptide chains. Biochem. Biophys. Res. Commun. 104,66-74.

Jones, J. L., Jones, J. J., Hanson, D. L., Dworkin, M. S. (1999). Surveillance for AIDS-defining opportunistic illnesses, 1992-1997. MMWR Morb. Mortal. Wkly Rep. 48, (SS-2), 1-22.

Kagan, H. M. (1986). Characterization and regulation of lysyl oxidase. In: Mecham, R. P. (ed.). Biology of Extacellular Matrix. Vol. 1, pp. 321-389, Academic Press, Orlando, Fla.

### Kagan, H. M. and Trackman, P. C. (1991). Properties and function of lysyl oxidase. Am. J. Respir. Cell Mol. Biol. 5,206-210.

Kagan, 1.M., Reddy, V. B., Narasimhan, N. and Csizsar, K. (1995). Catalytic property and structural components of lysyl oxidase. In: The Molecular Biology and Pathology of Elastic Tissue. pp.100-121, Wiley, Chichester (Ciba Foundation Symosium).

Kagan, H. M., Williams, M. A., Calaman, S. D. and Berkowitz, E. M. (1983). Histone H1 is a substrate for lysyl oxidase and contains endogenous sodium borotritide-reducible residues. Biochem. Biophysic. Res. Commun. 115,186-192.

Kaiser et al., (1998). Arthritis. Rheum., 41:623-633.

Karn, J. (1999). Tackling Tat. J. Mol. Biol. 293,235-254.

Keating, M. T. and Williams, L. T. (1988). Autocrine stimulation of intracellular PDGF receptors in v-sis-transformed cells.

Science 239,914-916.

Keck, R. G., Berleau, L., Harris, R. and Keyt, B. A. (1997). Disulfide structure of the heparin binding domain in vascular endothelial growth factor: characterization of posttranslational modification in VEGF. J. Biol. Chem. 344,103-113.

Kenyon, K., Contente, S., Trackman, P. C., Tang, J., Kagan, H. M. and Friedman, R. M. (1991). Lysyl oxidase and rrg messenger RNA. Science 253,802.

Kranz, A., Mattfeldt, T. and Waltenberger, J. (1999). Molecular mediators of tumor angiogenesis: enhanced expression and activation of vascular endothelial growth factor KDR in primary breast cancer. Int. J. Cance 84,293-298.

Krown, S. (1998). Clinical overview: issues in Kaposi's sarcoma therapeutics. J. Natl Cancer Inst. Monog. 23,59-63.

Kuivaniemi, H., Korhonen, R. M., Vaheri, A. and Kivirikko, K. I. (1986). Deficient production of lysyl oxidase in cultures of malignantly transformed human cells. FEBS lett. 195,261-264.

Lazarus, H. M., Cruikshank, W. W., Narasimhan, N., Kagan, H. M. and Center, D. M. (1994): Induction of human monocyte motility by lysyl oxidase. Matrix Biol. 14:727-731.

Leung, D. W., Cachianes, G., Kuang, W. J., Goeddel, D. V. and Ferrara, N. (1989). Vascular endothelial growth factor is a secreted angiogenic mitogen. Science. 246,1306-1309.

Li, P. F., Dietz, R. and von Harsdorf, R. (1997). Reactive oxygen species induce apoptosis of vascular smooth muscle cells. FEBS Lett. 404,249-252.

Li, W., Chou, I. N., Boak, A. and Kagan, H. M. (1995). Downregulation of lysyl oxidase in cadmium-resistant fibroblasts. Am. J. Respir. Cell Mol. Biol. 13,418-425.

Li, W., Liu, G., Chou, I-N. and Kagan, H. M. (2000). Hydrogen peroxide-mediated, lysyl oxidase-dependent chemotaxis of vascular smooth muscle cells. J. Cell. Biochem. (in press). Li, W., Nellaiappan, K., Strassmaier, T., Graham, L., Thomas, K. M. and Kagan, H. M. (1997). Localization and activity of lysyl oxidase within nuclei of fibrogenic cells. Proc. Natl. Acad. Sci. USA. 94:12817-12822.

Lo, Y. Y. C. and Cruz, T. F. (1995). Involvement of reactive oxygen species in cytokine and growth factor induction of c-fos expression in chondrocytes. J. Biol. Chem. 270,11727-11730.

Maglione, D., Guerriero, V., Viglietto, G., Delli-Bovi, P. and Persico, M. G. (1991). Isolation of a human placenta cDNA coding for a protein related to the vascular permeability factor. Proc. Natl. Acad. Sci. USA 88,9267-9271.

Maher, P. (1999). p38 Mitogen-activated protein kinases activation is required for fibroblast growth factor 2-stimulated cell proliferation but not diferentiation. J. Biol. Chem. 274.17491-17498.

Marasco, W. A., LaVecchio, J. and Winkler, A. (1999). Human anti-HIV-1 tat sFv intrabodies for gene therapy of advanced HIV-1-infection and AIDS. J. Immun. Meth. 231,223-238.

Marchio, S., Primo, L., Pagano, M., Palestro, G., Albini, A., Veikkola, T., Cascone, I., Alitano, K. and Bussolino, F. (1999). Vascular endothelial growth factor-C stimulates the migration and proliferation of Kaposi's sarcoma cells. J. Biol. Chem. 274,27617-27622.

Mason, I. J. (1994). The ins and outs of fibroblast growth factors. Cell 78, 547-552.

Masood, R., Flore, O., Cai, J. (1998). The VEGF family of proteins are autocrine growth and survival factors in AIDS-KS. Blood 91,10 (abstract).

Massod, R., Cai., J., Zheng, T., Smith, D. L., Naidu, Y. and Gill, P. (1997). Vascular endothelial growth factor/vascular permeability factor is an autocrine growth factor for AIDS-kaposi's sarcoma. Proc. Natl. Acad. Sci. USA 94,979-984.

Mauro, A., Bulfone, A., Turco, E. and Schiffer, D. (1991). Coexpression of platelet-derived growth factor (PDGF) B chain and PDGF B-type receptor in human gliomas. Child's Nerv. Syst. 7,432-436.

Mercola, D. (1992). Platelet-derived growth factor, transformation and antisense: models wanted. In Erickson, R. P. and Izant, J. (eds.) Gene Regulation by Antisense RNA and DNA. pp.329-353. UCLA Symposium on Molecular Biology. Keystone, Colo. Raven Press.

Mignatti, P., Morimoto, T. and Rifkin, D. B. (1992). Basic fibroblast growth factor, a protein devoid of secretory signal sequence, is released by cells via a pathway independent of the endoplasmic reticulum-Golgi complex. J. Cell Physiol. 151, 81-93.

Mihalcea, M. A., Smith, D. L., Monini, P., Sgadari, C., Ensoli, B. and Gill, P. S. (1999). Treatment update for AIDS-related Kaposi's sarcoma. AIDS 13, S215-S225.

Nakamura, S., Murakami-Mori, K., Rao, N., Weich, H. A. and Rajeev, B. (1997). Vascular endothelial growth factor is a potent angiogenic factor in AIDS-associated Kaposi's sarcoma-derived spindle cells. J. Immunol. 158,4992-5001.

The National Cancer Institute (2000). Kaposi's sarcoma (PDQ) treatment-health professionals. pp1-13, http://cancernet.nch.nih.gov/cgi-bin/srchcgi.exe.

Nellaiappan, K., Risitano, A., Liu, G., Nicklas, G. and Kagan, H. M. (2000). Fully processed lysyl oxidase catalyst translocates from the extracellular space into nuclei of aortic smooth-muscle cells. J. Cell. Biochem. 79,576-582.

Neufeld, G., Cohen, T., Gengrinovitch, S. and Poltorak, Z. (1999). Vascular endothelial growth factor (VEGF) and its receptors. FASEB J.13,9-22.

Nishimura, T., Utsunomiya Y., Hoshikawa, M., Ohuchi, H. and Itoh, N. (1999). Structure and expression of a novel human FGF, FGF-19, expressed in the fetal brain. Biochim. Biophys. Acta 1444,148-151.

Northfelt, D. W. and Volberding, P. A. (1991). AIDS-related Kaposi's sarcoma: clinical presentation, biology and therapy. Adv. Oncol. 7, 9-17.

Nugent, M. A. and Edelman E. R. (1992). Kinetics of Basic fibroblast growth factor binding to its receptor and heparan sulfate proteoglycan: a mechanism for cooperativity. Biochem 31, 8876-8883.

Ogawa, S., Oku, A., Sawano, A., Yamaguchi, S., Yazaki, Y. and Shibuya, M. (1998). A novel type of vascular endothelial growth factor, VEGF-E (NZ-7 VEGF), preferentially utilizes KDR/Flk-1 receptor and carries a potent mitotic activity without heparin-binding domain. J. Biol. Chem. 273,31273-31282.

Panckenko, M. V., Stetler-Stevenson, W. G., Trubetskoy, O. V., Gacheru, S. N. and Kagan, H. M. (1996). Metalloproteinase activity selected by fibrogenic cells in the processing of prolysyl oxidase. Potential role of procollagen C-proteinase. J. Biol. Chem. 271,7113-7119.

Park, J. E., Keller, G-A. and Ferrara, N. (1993). The vascular endothelial growth factor (VEGF) isoforms: differential deposition into the subepithelial extracellular matrix and bioactivity of extracellular matrix-bound VEGF. Mol. Biol. Cell 4,1317-1326.

Penault-Llorca, F., Bertucci, F., Adelaide, J., Parc, P., Coulier, F., Jacquemier, J., Birnbaum, D. and Delapeyriere, Q. (1995). Expression of FGF and FGF receptor genes in human breast cancer. Int. J. Cancer 61,170-176.

Potapova, O., Fakhrai, H., Baird, S. and Mercola, D. (1996).

Platelet-derived growth factor-B/v-sis confers a tumorigenic and metastatic phenotype to human T98G glioblastoma cells. Cancer Res. 56, 280-286.

Potorak, Z., Cohen, T., Sivan, R., Kandelis, Y., Spira, G., Vlodavsky, I., Keshet, E. and Neufeld, G. (1997). VEGF145, a secreted vascular endothelial growth factor isoform that binds to extracellular matrix. J. Biol. Chem. 272,7151-7158.

Prats, M., Kaghad, M., Prats, A. C., Klagsbrun, M., Lelias, J. M., Liauzun, P., Chalon, P., Tauber, F., Amalric, F., Smith, J. A. and Caput, D. (1989). High molecular mass forms of basic fibroblast growth factor are initiated by alternative CUG codons. Pro. Natl. Acad. Sci. USA 86,1836-1840.

Raines, E. W. and Ross, R. (1992). Compartmentalization of PDGF on extracellular binding sites dependent on exon-6-encoded sequences. J. Cell Biol. 116,533-543.

Reitz, Jr., M. S., Nerurkar, L. S. and Gallo, R. C. (1999). Perspective on Kaposi's sarcoma: Facts, concepts and conjectures. J. Natl. Cancer Inst. 91,1453-1458.

Rifkin, D. B. and Moscatelli, D. (1989). Recent develoments in the cell biology of basic fibroblast growth factor. J. Cell Biol. 109,1-6.

Rogelj, S., Weinberg, R. A., Fanning, P. and Klagsbrun, M. (1988). Basic fibroblast growth factor fused to a signal peptide transforms cells. Nature 331,173-175.

Ross, R., Gloset, J., Kariya, B. and Harker, L. (1974). A platelet-dependent serum factor that stimulates the proliferation of arterial smooth muscle cells in vitro. Proc. Natl. Acad. Sci. (USA) 71, 1207-1210.

Roy, S., Delling, U., Chen, C-H., Rosen, C. A. and Sonenberg, N. (1990). A bulge structure in HIV-1 TAR RNA is required for Tat binding and tat-mediated transactivation. Genes Dev. 4,1365-1373.

Rubartelli, A., Poggi, A., Sitia, R. and Zocchi, M. R. (1998). HIV-1 tat: a polypeptide for all seasons. mmun. Today 19,543-545

Ruben, S., Perkins, A., Purcell, R., Joung, K., Sia, R., Burghoff, R., Haseltine, W. A. and Rosen, C. A. (1989). Structural and functional characterization of human immunodeficiency virus tat protein. J. Virol. 63,1-8.

Ruker, R. B., Kosonen, T., Clegg, M. S., Mitchell, A. E., Rucker, B. R., Uriu-Hare, J. Y. and Keen, C. L. (1998). Copper, lysyl oxidase, and extracellular matrix protein cross-linking. Am. J. Clin. Nutr. 67,996S-1002S.

Samaniego, F., Markham, P. D., Gallo, R. C. and Ensoli, B. (1995). Inflammatory cytokines induce AIDS-Kaposi's sarcoma-derived spindle cells to produce and release basic fibroblast growth factor and to enhance Kaposi's sarcoma-like lesion formation in nude mice, J. Immunol. 154,3528-3592.

Samaniego, F., Markham, P. D., Gendelman, R., Gallo, R. C. and Ensoli, B. (1997). Inflammatory cytokines induce endothelial cells to produce and release basic fibroblast growth factor and to promote Kaposi's sarcoma-like lesions in nude mice, J. Immunol. 158,1887-1894.

Samaniego, F., Markham, P. D., Gendelman, R., Watanabe, Y., Kao, V., Kowalski, K., Sonnabend, A., Pintus, A., Gallo, R. C. and Ensoli, B. (1998). Vascular endothelial growth factor and basic fibroblast growth factor are expressed in Kaposi's sarcoma and synergize to induce angiogenesis, vascular permeability and KS lesion development: induction by inflammatory cytokines. Am. J. Pathol. 152,293-298.

Saux, C. J.-L, Tronecker, H., Bogic, L., Bryant-Greenwood, G. D., Boyd, C. D. and Csiszar, K. (1999). The LOXL2 gene encodes a new lysyl oxidase-like protein and is expressed at high levels in reproductive tissues. J. Biol. Chem. 274,12939-12944.

Scala, G., Ruocoo, M. R., Ambrosino, C., Mallardo, M., Giordano, V., Baldassarre, F., Dragonetti, E., Quinto, I. and Venuta, S. (1994). The expression of the interleukin 6 gene is induced by the human immunodeficiency virus 1 Tat protein. J. Exp. Med. 179,961-971.

Schlessinger, J. Lax, I. and Lemmon, M. (1995). Regulation of growth factor activation by proteoglycans: what is the role of low affinity receptors. Cell 83, 357-360.

Shweiki, D., Itin, A., Soffer, D. and Keshet, E. (1992). Vascular endothelial growth factor induced by hypoxia may mediate hypoxia-initiated angiogenesis. Nature (London) 359,843-845.

Smits, A., Funa, K., Vassbotn, F. S., Beausang-Linder, M., of Ekenstam, F., Heldin, C-H., Westermark, B. and Nister, M. (1992). Expression of platelet-derived growth factor and its receptors in proliferative disorders of fibroblastic Am. J. Pathol. 140,639-648.

Soker, S., Takashima, S., Miao, H. Q., Neufeld, G. and Klagsbrun, M. (1998). Neuropilin-1 is expressed by endothelial and tumor cells as an isoform-specific receptor for vascular endothelial growth factor. Cell 92,735-745.

Sperinde, G. V. and Nugent, M. A. (1998). Heparan sulfate proteoglycans control bFGF processing in vascular smooth muscle cells. Biochem. 37,13153-13164.

Sporn, M. B. and Roberts, A. B. (1985). Autocrine growth factors and cancer. Nature (London) 313,745-747).

Stachowiak, E. K., Maher, P. A. Tucholski, J., Mordechai, E., Joy, A., Moffett, J., Coons, S. and Stachowiak, M. K. (1997). Nuclear accumulation of fibroblast growth factor receptors in human glial cells-association with cell proliferation. Oncogene 14,2201-2211.

Stachowiak, M. K., Moffett, J., Joy, I., Puchacz, E., Florkiewicz, R. and Stachowiak, E. K. (1996). Regulation of bFGF gene expression and subcelluar distribution of bFGF protein in adrenal medullary cells. J. Cell Biol. 127,203-223.

Sundaresan M, Yu Z-X, Ferrans V J, Irani K, Finkel T (1995): Requirement for generation of H₂O₂ for platelet-derived growth factor signal transduction. Science 270:296-299.

Terman, B. I., Dougher-Vermazen, M., Carrion, M. E., Dimitrov, D., Armellino, D. C., Gospodarowicz, D. and Bohlen, P., (1992). Identification of the KDR tyrosine kinase as a receptor for vascular endothelial growth factor. Biochem. Biophys. Res. Commun. 187,1579-1586.

Thompson et al., (1998). J. Clin. Endocrinol. Metab., 83:1336-1341.

Tischer, E., Mitchell, R., Hartman, T., Silva, M., Gospodarowicz, D., Fiddes, J. C. and Abraham, J. A. (1991). The human gene for vascular endothelial growth factor. Multiple protein forms are encoded through alternative exon splicing. J. Biol. Chem. 266,11947-11954.

Trackman, P. C., Bedell-Hogan, D., Tang, J. and Kagan, H. M. (1992). Post-translational glycosylation and proteolytic processing of a lysyl oxidase precursor. J. Biol. Chem. 267,8666-8671.

Trackman, P. C., Zoski, C. G. and Kagan, H. M. (1981). Development of a peroxidase-coupled fluorometric assay for lysyl oxidase. Anal Biochem. 113,336-342.

Ullrich, A. and Schlessinger, J. (1990). Signal transduction by receptors with tyrosine kinase activity. Cell 61,203-212.

Vogel, J., Hinrichs, S. H., Reynolds, R. K., Luciw, P. A. and Jay, G. (1988). The HIV tat gene induces dermal lesions resembling Kaposi's sarcoma in transgenic mice. Nature 335,601-661.

Volm, M., Koomaji, R., Mattern, J. and Stammler, G. (1997). Prognostic value of basic fibroblast growth factor and its receptor (FGFR1) in patients with non-small cell lung carcinomas. Euro. J. Cancer 33,691-693.

Weindel, K., Marme, D. and Weich, H. A. (1992). AIDS-associated Kaposi's sarcoma cells in culture express vascular endothelial growth factor. Biochem Biophys. Res. Commun 183,1167-1174.

Wellstein, A. and Lippman, M. E. (1991). Fibroblast growth factor and breast cancer. In Broder, S. (ed.) Molecular Foundations of Oncology. pp.403-417, Williams & Wilkins, Baltimore, Md.

Westendrop, M. O., Frank, R., Ochsenbauer, C., Stricker, K., Dhein, J., Walczak, H., Debatin, K-M. and Krammer, P. H. (1995). Sensitization of T cells to CD95-mediated apoptosis by HIV-1 Tat and gp120. Nature 275,497-500.

Xerri, L., Haussoun, J., Planche, J., Guigou, V., Grob, J-J., Parc, P., Birnbaum, D. and deLapeyriere, O. (1991). Fibroblast growth factor gene expression in AIDS-Kaposi's sarcoma detected by in situ hybridization. Am. J. Pathol. 138,9-15.

Yayon, A. and Klagsbrun, M. (1990). Autocrine transformation by chimeric signal peptide-basic fibroblast growth factor: reversal by suramin. Proc. Natl. Acad. Sci. (USA) 87,5346-5350.

Yen-Moore, A., Hudnall, S. D., Rady, P. L., Wagner, Jr., R. F., Moore, T. O., Memar, O., Hughes, T. K., Tyring, S. K. (2000). Differential Expression of the HHV-8 vGCR cellular homolog gene in AIDS-associated and classic Kaposi's sarcoma: potential role of HIV-1 Tat. Virol. 267,247-251.

Zauli, G., Gibellini, D., Caputo, A., Bassini, A., Negrini, M., Monne, M., Mazzoni, M. and Capitani, S. (1995). The human immunodeficiency virus type 1 Tat protein upregulates Bcl-2 gene expression in Jurkat T-cell lines and primary peripheral blood mononuclear cells Blood. 86,3823-3834.

Zhang, H-T., Scott, P., Morbidelli, L., Peak, S., Moore, J., Turley, H., Harris, A. L., Ziche, M. and Bicknell, R. (2000). The 121 amino acid isoform of vascular endothelial growth factor is more strongly tumorigenic than other splice variants in vivo. Brit. J. Cancer 83,63-68.

Equivalents

While the present invention has been described in conjunction with a preferred embodiment, one of ordinary skill, after reading the foregoing specification, will be able to effect various changes, substitutions of equivalents, and other alterations to the compositions and approaches set forth herein. It is therefore intended that the protection granted by Letters Patent hereon be limited only by the definitions contained in the appended claims and equivalents thereof.

What is claimed is:

1. A therapeutic composition for prophylaxis and treatment of a condition associated with an abnormal cellular proliferation, said composition comprising:
an effective amount of a therapeutically active portion of an inhibitor in a pharmaceutically acceptable inert carrier substance;
wherein said inhibitor inactivates a growth factor;
wherein said inhibitor oxidizes said growth factor at lysine residues; and
wherein said amount of said inhibitor is effective in preventing and treating a condition associated with said abnormal cellular proliferation.

2. A therapeutic composition for prophylaxis and inhibition of a condition associated with angiogenesis comprising:
an effective amount of a therapeutically active portion of an inhibitor in a pharmaceutically acceptable inert carrier substance;
wherein said inhibitor inactivates an angiogenic factor;
wherein said inhibitor oxidizes said angiogenic factor at lysine residues; and
wherein said amount of said inhibitor is effective in inhibiting said angiogenesis.

3. A therapeutic composition for prophylaxis and inhibition of a condition associated with a microorganism infection comprising:
an effective amount of a therapeutically active portion of an inhibitor in a pharmaceutically acceptable inert carrier substance;
wherein said inhibitor inactivates a transactivator for replication of said microorganism;
wherein said inhibitor oxidizes said transactivator at lysine residues; and
wherein said amount of said inhibitor is effective in inhibiting microorganism replication.

4. The therapeutic composition of any of claims 1, 2, or 3, wherein said inhibitor is lysyl oxidase.

5. The therapeutic composition of any of claims 1, 2, or 3, wherein said inhibitor is a homologue of lysyl oxidase.

6. The therapeutic composition of any of claims 1, 2, or 3, wherein said lysyl oxidase is purified from bovine or other mammalian connective tissue.

7. The therapeutic composition of any of claims 1, 2, or 3, wherein said inhibitor is co-administered with a compound selected from the group consisting of an antineoplastic agent, a cytokine, a hormone, and a copper ion.

8. The therapeutic composition of any of claims 1, 2, or 3, wherein said condition is cancer.

9. The therapeutic composition of any of claims 1, 2, or 3, wherein said condition is associated with a disease selected from the group consisting of breast cancer, colon cancer, renal cancer, prostate cancer, ovarian cancer, lung cancer, brain cancer, skin cancer, embryo carcinoma, teratocarcinoma, germ cell tumor, uterine cancer, osteocarcoma, fibrosacoma, melanoma, AIDS-associated malignancies, angiogenic diseases, other tumors, and hyperplastic diseases with or without inflammation.

10. The therapeutic composition of any of claims 1, 2, or 3, wherein said abnormal cellular proliferation is a tumor, lesion, or wound.

11. The therapeutic composition of claim 1, wherein said abnormal cellular proliferation is a human cell proliferation.

12. The therapeutic composition of claim 3, wherein said condition associated with a microorganism infection is AIDS.

13. The therapeutic composition of claim 3, wherein said microorganism is HIV-1.

14. The therapeutic composition of claim 3, wherein said transactivator for the replication of said microorganism is HIV-1 Tat.

15. A kit comprising a therapeutic composition of any of claims 1, 2, or 3 and instructions for use thereof.

16. A method of treating a patient believed to be at risk of suffering from a disease associated with abnormal cellular proliferation, said method comprising the steps of:
providing said patient; and
administering to said patient an effective amount of the therapeutic composition of any of claims 1 or 2.

17. A method of modulating cellular proliferation and angiogenesis, said method comprising the steps of:
contacting mitogenic and angiogenic factor with an inhibitor in an effective amount to modulate cell proliferation, whereby said inhibitor is a therapeutic composition of any of claims 1 or 2; and
determining regulation of cell proliferation.

18. A method of treating a patient believed to be at risk of suffering from a condition associated with a microorganism infection, said method comprising the steps of:
providing said patient; and
administering to said patient an effective amount of the therapeutic composition of claim 3.

19. The method of any of claims 16, 17, or 18, wherein said disease is selected from the group consisting of breast cancer, colon cancer, renal cancer, prostate cancer, ovarian cancer, lung cancer, brain cancer, skin cancer, embryo carcinoma, teratocarcinoma, germ cell tumor, uterine cancer, osteocarcoma, fibrosacoma, malanoma, AIDS-associated malignancies, angiogenic diseases, other tumors and hyperplastic diseases with or without inflammation.

20. The method of any of claims 16 or 18, said method comprising the additional step of determining treatment progress by tissue biopsy.
